(19) United States
(12) Patent Application Publication
(10) Pub. No.: US 2014/0329915 A1
(43) Pub. Date: Nov. 6, 2014

(54) HYDROGEL ADAPTED FOR TREATMENT OF ACUTE DERMAL WOUNDS

(71) Applicant: Halscion, Inc., Duluth, GA (US)

(72) Inventors: Tien Nguyen, Daly City, CA (US); Kathleen Prudhomme, Atlanta, GA (US); Ronald Yamamoto, San Francisco, CA (US); Adam G. Lowe, Snellville, GA (US); Andrew Michael Green, Cumming, GA (US)

(73) Assignee: Halscion, Inc., Duluth, GA (US)

(21) Appl. No.: 14/266,407

(22) Filed: Apr. 30, 2014

Related U.S. Application Data
(60) Provisional application No. 61/818,238, filed on May 1, 2013.

(51) Int. Cl. A61K 47/42; A61K 47/36

(52) U.S. Cl. CPC A61K 47/42; A61K 47/36
USPC 514/774

(57) ABSTRACT

The present invention provides compositions and methods useful in the treatment of wounds, particularly in reducing or preventing scar formation, particularly hypertrophic scar or keloid formation. The invention thus further provides methods of treatment, including methods useful in hypertrophic scar or keloid revision as well as prophylactic, scar inhibiting methods.

### FIELD OF THE INVENTION

The present invention relates to compositions adapted for treatment of dermal wounds, methods for such treatment, and methods for preparing such compositions. In particular, the compositions can be defined by a hydrogel stability factor according to which the compositions can be particularly effective for use in dermal wounds, preferentially in a manner effective to promote healing with reduced scarring, particularly keloid formation.

### BACKGROUND OF THE INVENTION

There are many situations in medicine where a wound does not heal properly, such as from compromised wound healing that delays or prevents resolution of a wound. There are further wound healing events, however, that can limit resulting function or cosmesis. Exemplary undesired results include hyperplastic responses that produce extensive scarring, keloids, or wound contracture that compromises function and mobility.

Hypertrophic scars occur when the body overproduces collagen, which causes the scar to be raised above the surrounding skin. Hypertrophic scars often take the form of a red raised lump on the skin and usually occur within four to eight weeks following wound infection or wound closure with excess tension and/or other traumatic skin injuries. Keloid formation in particular is a very challenging wound healing problem. Keloids are defined as benign fibrous (fibroblastic or myofibroblastic) proliferations resulting in soft tissue tumors. The benign hyper-proliferative growth of dense fibrous tissue in keloids develops from an abnormal healing response to a cutaneous injury and is dissimilar to normal wound healing and scarring, including hypertrophic scarring. These differences manifest in cellular processes, collagen production and deposition, continued growth beyond the boundaries of the original wound, and a high recurrence rate after excision. Unlike normal scars or hypertrophic scars, keloids contain fibroblasts that overproduce type I procollagen, VEGF, TGFβ1/β2, PDGF-α receptors, and have reduced growth factor requirements, with either lower rates of apoptosis or a down-regulation of apoptotic genes (Robles, et al., Clinics in Dermatology, 2007). These aberrant fibroblast processes have demonstrated increased production of collagen and extracellular matrix in vitro and in vivo. X-ray diffraction examination of normal, hypertrophic, and keloid scars demonstrates that rather than the collagen fibrils running parallel to the scar line (normal scar) or slightly aligned to the scar line (hypertrophic scars), the collagen fibrils of keloid scars present no specific orientation of the collagen at all (Koonin eta al., S. A. Medical Journal 1964). Additionally, this collagen has been termed “keloid collagen” since the deposition pattern, mixture of collagen types (greater abundance of type III later replaced by type I), and overabundance are unlike other tissue or scar types (Cheng et al. African Journal of Biotechnology 2011). Finally, unlike normal scars or hypertrophic scars, the propensities for recurrence in keloid scars have been reported at 45-100%, and keloids are resistant to known treatments used for scars or hypertrophic scars (Robles, et al., Clinics in Dermatology 2007).

Compositions to promote wound healing have been described with the use of collagen, the main structural protein of the body. In particular, compositions of collagen combined with a glycosaminoglycan, the structural polysaccharides of the body, have also been described to promote wound healing or act as tissue templates for wound repair. For example, U.S. Pat. No. 4,837,024 describes promoting wound healing by contacting a surface of a wound with a suspension of particles of collagen and a glycosaminoglycan. U.S. Pat. No. 4,280,954 describes a composite material containing collagen and a mucopolysaccharide (glycosaminoglycan) that is useful as a degradable surgical prosthesis such as a synthetic skin. Compositions using denatured collagen have also been described in wound healing compositions with polysaccharides. U.S. Pat. No. 6,261,587 and U.S. Pat. No. 6,713,079 describe compositions of gelatin and dextran or heparin to be used to stimulate vascularization and promote wound healing. There still remains a need for methods and compositions useful in dermal wound treatment, specifically acute dermal wound treatment, and particularly in addressing keloids and hypertrophic dermal healing.

### SUMMARY OF THE INVENTION

The present invention provides stable hydrogel compositions that can be useful in wound treatment to promote healing, and to particularly promote healing in a manner whereby hypertrophic wound healing and/or scar formation is reduced or limited. The invention further provides methods of forming hydrogel compositions that are stabilized against phase separation and structural rearrangement, methods of using such compositions, and articles of manufacture incorporating such compositions. The inventive methods and compositions can be particularly useful to reduce or limit scarring including but not limited to post-surgical scars, hypertrophic scars, scars resulting from trauma or burns, and keloids.

In various embodiments, the present disclosure relates to a method of preparing a stable hydrogel composition. In an example embodiment, the method can comprise mixing gelatin and a polymeric carbohydrate in an aqueous medium at a first temperature to form a liquid hydrogel composition wherein the gelatin comprises about 60% by weight or greater of the total weight of the combination of the gelatin and the polymeric carbohydrate present in the composition. The method further can comprise cooling the liquid hydrogel composition with constant mixing to a holding temperature that is less than the first temperature and greater than the gelation temperature at which the liquid hydrogel composition transitions to a solid or semi-solid hydrogel composition. The method also can comprise further cooling the liquid hydrogel composition to the gelation temperature to transition the liquid hydrogel composition to a stable, solid or semi-solid hydrogel composition.

The first temperature at which mixing is carried out can be, for example, about 45° C. or greater, more particularly about 46° C. to about 70° C. The holding temperature can be at least about 5° C. less than the first temperature. In further embodiments, the holding temperature can be within about 7° C. of the gelation temperature. The gelation temperature can be, for example, less than about 35° C. In some embodiments, further cooling to the gelation temperature can be carried out in a time of less than about 2 hours. The further cooling particularly can comprise cooling the stable, solid or semi-solid hydrogel composition to a storage temperature. The storage temperature can be about 1° C. to about 12° C. Additionally, the methods further may comprise lyophilizing the stable hydrogel composition.

In specific embodiments, the aqueous medium used to form the hydrogel can be defined by its salt concentration. For example, an aqueous medium for forming the hydrogel can have an osmolality of less than about 400 mOsm/kg. In further embodiments, the aqueous medium can have an osmolality of about 25 mOsm/kg to about 375 mOsm/kg. In other embodiments, the aqueous medium can have a phosphate ion concentration of no greater than about 20 mM. Similarly, the aqueous medium can have a carbonate ion concentration of no greater than about 20 mM. In some embodiments, the aqueous medium can comprise Medium 199.

The hydrogel composition formed according to the above methods may be further characterized by its composition and properties. For example, the total concentration of the gelatin and the polymeric carbohydrate in the stable hydrogel composition can be about 50 mg/mL to about 400 mg/mL.

In specific embodiments, the stable hydrogel composition formed according to the above methods may be defined by its intrinsic stability against phase separation and structural rearrangement that has been found to arise from the specific methods of preparation of the hydrogel composition. Such intrinsic stability can be characterized in relation to ultrasonic evaluation that reveals the ability of the hydrogel to resist phase separation and structural rearrangement. Thus, the stable hydrogel can be identified based upon exhibiting an ultrasonic attenuation hydrogel stability factor. For example, the stable hydrogel composition according to the present disclosure can exhibit an ultrasonic attenuation (U_{A}) hydrogel stability factor of 0.4 for at least 500 minutes when tested at a temperature of 35° C. and a frequency of 2-8 MHz. This stability factor indicates that under the test conditions, the ultrasonic attenuation of the tested hydrogel changes by less than 40% over the test period. In further embodiments, the stable hydrogel composition can exhibit a U_{A} hydrogel stability factor of 0.5 for at least 600 minutes when tested at a temperature of 35° C. and a frequency of 2-8 MHz. In further embodiments, the stable hydrogel composition can be defined by one or more of: a U_{A} hydrogel stability factor of 0.3 for at least 500 minutes when tested at a temperature of 35° C. and a frequency of 2.7 MHz; a U_{A} hydrogel stability factor of 0.4 for at least 600 minutes when tested at a temperature of 35° C. and a frequency of 2.7 MHz; a U_{A} hydrogel stability factor of 0.2 for at least 500 minutes when tested at a temperature of 35° C. and a frequency of 5.1 MHz; a U_{A} hydrogel stability factor of 0.3 for at least 600 minutes when tested at a temperature of 35° C. and a frequency of 5.1 MHz; a U_{A} hydrogel stability factor of 0.35 for at least 500 minutes when tested at a temperature of 35° C. and a frequency of 7.8 MHz; and a U_{A} hydrogel stability factor of 0.5 for at least 600 minutes when tested at a temperature of 35° C. and a frequency of 7.8 MHz.

In further embodiments, the present disclosure provides a stable hydrogel composition. The stable hydrogel composition preferably is a hydrogel that has been prepared according to the methods described herein, and such hydrogel composition can be characterized by its further properties. For example, the stable hydrogel composition can have a flow rate of about 10 μL/s or greater when forced from a syringe through a ⅝ inch long 25 gauge needle under a syringe plunger pressure of 5 N at a temperature of 35° C. to 39° C. The stable hydrogel composition can have a viscosity of about 1.5 Pa-s or less at a temperature of 35° C. to 39° C. The stable hydrogel composition can have a fibronectin binding activity of about 3 nmol/mg or greater. The stable hydrogel composition can have a residence time in the dermal or subdermal tissue of a mammal of about 3 days or greater.

In still further embodiments, the present disclosure can relate to a hydrogel composition that has been reconstituted from a lyophilized form. Although lyophilized materials are known in the art, it has not heretofore been recognized that it can be difficult to reconstitute a lyophilized hydro gel matrix comprising gelatin and a polymeric carbohydrate in a form that is suitable for injection, particularly through a small gauge needle, and that can be quickly and easily reconstituted to the flowable form.

In exemplary embodiments, the present disclosure thus can provide a flowable, injectable composition comprising gelatin and a polymeric carbohydrate, the composition being a hydrogel reconstituted from a lyophilized form and further including two or more additives selected from the group consisting of surfactants, hygroscopic excipients, and bulking agents. Preferably, the gelatin comprises about 60% by weight or greater of the total weight of the combination of the gelatin and the polymeric carbohydrate present in the reconstituted composition. In further embodiments, the flowable, injectable reconstituted composition has a flow rate of about 25 μL/s or greater when forced from a syringe through a ⅝ inch long 25 gauge needle under a syringe plunger pressure of 5 N at a temperature of 35° C. to 39° C.

The two or more additives particularly may comprise any of: i) a surfactant and a hygroscopic excipient; ii) a surfactant and a bulking agent; iii) a hygroscopic excipient and a bulking agent; and iv) a surfactant, a hygroscopic excipient, and a bulking agent. In specific embodiments, the two or more additives comprise any of: i) a polysorbate and a polyol; ii) a polysorbate and a salt; iii) a polysorbate and a sugar; iv) a polyol and a sugar; v) a salt and a sugar; vi) a polysorbate, a polyol, and a sugar; and vii) a polysorbate, a salt, and a sugar. In further embodiments, the two or more additives comprise any of: i) Tween and glycerol; ii) Tween and NaCl; iii) Tween and a disaccharide; iv) glycerol and a disaccharide; v) NaCl and a disaccharide; vi) Tween, glycerol, and a disaccharide; and vii) Tween, NaCl, and a disaccharide. The two or more additives in particular may comprise a surfactant and a hygroscopic excipient in a ratio of about 1:20 to about 20:1. The two or more additives also may comprise a surfactant and a bulking agent in a ratio of about 1:40 to about 10:1. The two or more additives further comprise a hygroscopic excipient and a bulking agent in a ratio of about 1:10 to about 10:1.

Preferably, the flowable, injectable reconstituted composition has a viscosity of about 1.5 Pa-s or less at a temperature of 35° C. to 39° C. The flowable, injectable reconstituted composition can be adapted to transition to a solid or semi-solid at temperatures less than 35° C. Preferably, the solid or semi-solid reconstituted composition has a residence time in dermal or subdermal tissue of about 3 days or greater.

In further embodiments, the present disclosure can provide a method for preventing or reducing cutaneous scarring, or an effect thereof, arising from a cutaneous wound. In particular, the method can comprise applying a composition as described herein to the cutaneous wound. The method further can comprise closing the wound with a closure selected from the group consisting of sutures, staples, glue, and combinations thereof. The applying step can comprise injecting the flowable, injectable liquid composition into dermal or subdermal tissue in proximity to the cutaneous wound. In specific embodiments, the cutaneous wound can be a surgical wound, for example a wound remaining after excision of at least a portion of a pre-existing scar, which can be a keloid, hypertrophic scar, or burn-related scar. The applying step may comprise topical application of the matrix composition. Preferably, keloid formation or formation of hypertrophic scarring is prevented or reduced using the present methods. For example, an effect of scarring that can be prevented or reduced can include pain, itching, discoloration, abnormal stiffness, abnormal thickness, surface irregularity, and combinations thereof. In particular embodiments, the applying step can comprise applying about 0.1 mL to about 10 mL of matrix composition per 2.5 cm of wound margin.

Further methods of preparing a hydrogel composition according to the present disclosure can include reconstituting a lyophilized form of the hydrogel to form a reconstituted composition as described herein. In an exemplary embodiment, a method of preparing a hydrogel composition can comprise providing a lyophilized composition comprising gelatin and a polymeric carbohydrate, the gelatin comprising about 60% by weight or greater of the total weight of the combination of the gelatin and the polymeric carbohydrate present in the lyophilized composition, and reconstituting the lyophilized composition with an aqueous reconstitution fluid to form the hydrogel composition such that the reconstituted hydrogel composition includes two or more additives selected from the group consisting of surfactants, hygroscopic excipients, and bulking agents, and such that the total concentration of the gelatin and the polymeric carbohydrate in the reconstituted hydrogel composition is about 50 mg/mL to about 400 mg/mL. In particular embodiments, the aqueous reconstitution fluid in contact with the lyophilized composition can comprise about 0.01% to about 4% by weight of the surfactant. Further, the aqueous reconstitution fluid in contact with the lyophilized composition can comprise about 0.01% to about 4% by weight of the hygroscopic excipient. Still further, the aqueous reconstitution fluid in contact with the lyophilized composition can comprise about 0.01% to about 4% by weight of the bulking agent. Additionally, the aqueous reconstitution fluid in contact with the lyophilized composition can comprise about 0.05% to about 6% by weight of the additives in total.

In various embodiments, the two or more additives can comprise any of: i) a surfactant and a hygroscopic excipient; ii) a surfactant and a bulking agent; iii) a hygroscopic excipient and a bulking agent; and iv) a surfactant, a hygroscopic excipient, and a bulking agent. In other embodiments, the two or more additives can comprise any of: i) a polysorbate and a polyol; ii) a polysorbate and a salt; iii) a polysorbate and a sugar; iv) a polyol and a sugar; v) a salt and a sugar; vi) a polysorbate, a polyol, and a sugar; and vii) a polysorbate, a salt, and a sugar. Further, the two or more additives can comprise any of: i) Tween and glycerol; ii) Tween and NaCl; iii) Tween and a disaccharide; iv) glycerol and a disaccharide; v) NaCl and a disaccharide; vi) Tween, glycerol, and a disaccharide; and vii) Tween, NaCl, and a disaccharide. In particular, the two or more additives can comprise a surfactant and a hygroscopic excipient in a ratio of about 1:20 to about 20:1; the two or more additives can comprise a surfactant and a bulking agent in a ratio of about 1:40 to about 10:1; and/or the two or more additives can comprise a hygroscopic excipient and a bulking agent in a ratio of about 1:10 to about 10:1.

In additional embodiments, the present disclosure further relates to articles of manufacture, such as kits, that can provide one or more components useful in forming a hydrogel composition as described herein. In an exemplary embodiment, a kit can comprise: a first container housing a lyophilized composition comprising gelatin and a polymeric carbohydrate, the gelatin comprising about 60% by weight or greater of the total weight of the combination of the gelatin and the polymeric carbohydrate present in the lyophilized composition; a second container housing a reconstitution material; and instructions for combining the contents of the first container and the second container to form a reconstituted hydrogel composition useful for treatment of an acute dermal wound. In particular, the combined contents of the first container and the second container include two or more additives selected from the group consisting of surfactants, hygroscopic excipients, and bulking agents.

In further embodiments, the first container can include at least one of the additives. The second container can include the two or more additives. The second container can include an aqueous reconstitution fluid. The aqueous reconstitution fluid can include the two or more additives. In various embodiments, the aqueous reconstitution fluid can comprise about 0.01% to about 4% by weight of the surfactant; the aqueous reconstitution fluid can comprise about 0.01% to about 4% by weight of the hygroscopic excipient; the aqueous reconstitution fluid can comprise about 0.01% to about 4% by weight of the bulking agent; and/or the aqueous reconstitution fluid can comprise about 0.05% to about 6% by weight of the additives.

In further embodiments, the two or more additives can comprise any of: i) a surfactant and a hygroscopic excipient; ii) a surfactant and a bulking agent; iii) a hygroscopic excipient and a bulking agent; and iv) a surfactant, a hygroscopic excipient, and a bulking agent. The two or more additives can comprise any of: i) a polysorbate and a polyol; ii) a polysorbate and a salt; iii) a polysorbate and a sugar; iv) a polyol and a sugar; v) a salt and a sugar; vi) a polysorbate, a polyol, and a sugar; and vii) a polysorbate, a salt, and a sugar. The two or more additives can comprise any of: i) Tween and glycerol; ii) Tween and NaCl; iii) Tween and a disaccharide; iv) glycerol and a disaccharide; v) NaCl and a disaccharide; vi) Tween, glycerol, and a disaccharide; and vii) Tween, NaCl, and a disaccharide. The two or more additives can comprise a surfactant and a hygroscopic excipient in a ratio of about 1:20 to about 20:1. The two or more additives can comprise a surfactant and a bulking agent in a ratio of about 1:40 to about 10:1. The two or more additives can comprise a hygroscopic excipient and a bulking agent in a ratio of about 1:10 to about 10:1.

A kit further can include further components useful in forming or delivering the hydrogel composition. For example, the kit further can comprise a connector adapted for one or both of sterile transfer of the contents of one of the first container into the second container and sterile transfer of the contents of the second container into the first container. At least one of the first container and the second container can be a syringe. The kit further can comprise a needle adapted for attachment to the syringe. In particular, the needle can be a 23 gauge through 27 gauge needle.

### BRIEF DESCRIPTION OF THE FIGURES

FIG. 1 is a graph showing a differential scanning calorimetry curve for exemplary compositions according to the present disclosure;

FIG. 2 is a graph showing physical properties of compositions according to exemplary embodiments of the present disclosure;

FIG. 3 is a graph showing physical properties of compositions according to further embodiments of the present disclosure relative to the gelatin concentration of the compositions;

FIG. 4 is a graph showing relative ultrasonic attenuation of a hydrogel composition prepared according to the method of present Example 1 at three different frequencies with a water reference;

FIG. 5 is a graph showing relative ultrasonic attenuation of a hydrogel composition prepared according to the method of present Example 2 at three different frequencies;

FIG. 6 is a graph showing relative ultrasonic velocity of a hydrogel composition prepared according to the method of present Example 1 and a hydrogel composition prepared according to present Example 2;

FIG. 7 illustrates a kit according to an exemplary embodiment of the present disclosure comprising a plurality of containers each containing components for preparation of a hydrogel composition;

FIG. 8a is a graph showing the phase changes of a composition prepared according to an embodiment of the present disclosure when incubated at a temperature of 40° C. for a time of 24 hours;

FIG. 8b is a graph showing the phase changes of a hydrogel composition prepared according to an embodiment of the present disclosure when incubated at a temperature of 40° C. for a time of 24 hours;

FIG. 8c is a graph showing the phase changes of a composition prepared without an intermediate cooling step when incubated at a temperature of 40° C. for a time of 24 hours;

FIG. 8d is a graph showing the phase changes of a further composition prepared without an intermediate cooling step when incubated at a temperature of 40° C. for a time of 24 hours;

FIG. 9a is a graph showing the phase changes of a composition prepared according to an embodiment of the present disclosure when incubated, at a temperature of 50° C. for a time of 24 hours;

FIG. 9b is a graph showing the phase changes of a further composition prepared according to an embodiment of the present disclosure when incubated at a temperature of 50° C. for a time of 24 hours;

FIG. 9c is a graph showing the phase changes of a composition prepared without an intermediate cooling step when incubated at a temperature of 50° C. for a time of 24 hours; and

FIG. 9d is a graph showing the phase changes of a further composition prepared without an intermediate cooling step when incubated at a temperature of 50° C. for a time of 24 hours.

### DETAILED DESCRIPTION OF THE INVENTION

The present invention will now be described more fully hereinafter with reference to exemplary embodiments thereof. These exemplary embodiments are described so that this disclosure will be thorough and complete, and will fully convey the scope of the invention to those skilled in the art. Indeed, the invention can be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The present disclosure relates to compositions that are beneficial for use in the treatment of wounds, particularly wounds to the skin (i.e., the dermis). While the average core body temperature of a human is 37° C. (+/−0.5° C.), skin temperatures can be significantly lower and typically are in the range of about 33° C. (+/−1° C.). The present disclosure describes compositions formed of gelatin and a polymeric carbohydrate that can be provided in a variety of forms and thus can be particularly useful in treating skin wounds. More specifically, the gelatin and the polymeric carbohydrate can be combined such that the interaction of the gelatin and the carbohydrate forms a solid or semi-solid gel matrix composition at temperatures that are about equal to or below average skin temperature. As more fully discussed below, the composition can be a solid or semi-solid material at a temperature that is less than 35° C.

In the dermal or subdermal tissue, the composition is substantially in the form of a solid or semi-solid gel matrix that modulates wound healing by its physical presence and potential interactions of the gelatin and the polymeric carbohydrate during the wound healing and wound maturation process. The composition of the present disclosure biodegrades and/or is physiologically resorbed by the tissues surrounding the implanted matrix composition. Thus, the solid or semi-solid matrix composition has a defined residence time during which the composition improves wound healing and slowly degrades. As further described herein, the composition has a hydrogel stability factor that defines an ability of the composition to retain the hydrogel structure that arises from the interactions of the gelatin and the polymeric carbohydrate. This is unlike previous hydrogels that separate into constituent components under dermal conditions and thus lack necessary characteristics to modulate wound healing in the manner possible according to the present disclosure. The composition beneficially can be provided in a dried (e.g., powder) form that is particularly useful for long-term storage of the composition. Such powder compositions can be reconstitutable using a variety of physiologically acceptable solvents and additives.

While the invention is not bound by a particular mechanism, it is believed that the combined gelatin and polymeric carbohydrate provides an environment that is similar to the native wound healing environment (e.g., in the presence of collagen and glycosaminoglycans). Such an environment can reduce or limit hypertrophic wound healing, including hypertrophic scar formation and keloid formation.

The compositions of the present disclosure provide the advantage of being a flowable (and injectable) liquid at temperatures that are only slightly above average dermal temperature and thus can be injected without thermal damage to the interfacing tissues. The compositions provide the further advantage, however, of transitioning into a solid or semi-solid gel matrix at about average dermal temperatures and maintaining the hydrogel structure for an extended period of time. The present composition thus can exhibit sufficient persistence at the vicinity of a dermal wound to have beneficial effect during the wound healing process that is not believed to occur with heretofore known hydrogel compositions. Preferably, the composition can be degradable but can persist during the acute wound healing period, e.g., approximately two weeks, but can remain for a longer period of time to benefit collagen maturation and wound remodeling. The composition also can be particularly useful in treatment of acute dermal wounds. The composition also can be useful for the treatment of keloid scars, hypertrophic scars, burn-related scars, or for the prophylactic treatment of surgical wounds, particularly in patients prone to scar formation. Patients falling into such categories can be identified based upon past history of scar formation—e.g., hypertrophic scarring or keloid formation. Additionally, patients can be identified based upon characterization into a group that is recognized in the art as being at increased risk for keloid formation (i.e., darkly pigmented skin). For example, there is a fifteen times higher frequency of keloid occurrence in highly pigmented people as compared to less pigmented people. More specifically, persons of African descent are recognized as being at increased risk of keloid occurrences.

The compositions of the present disclosure can be particularly characterized in relation to specific properties thereof. For example, during production of gelatin, a variety of chemical processes are used to degrade the collagen that can also degrade the fibronectin and polymeric carbohydrate binding properties of the resultant gelatin. To have the desired properties, the composition is preferred to comprise a gelatin with intact fibronectin binding activity. To this end, it can be preferential to use a gelatin of a defined molecular mass. Specifically, the gelatin can have an average molecular mass of about 75,000 or greater, about 80,000 Da or greater, about 100,000 Da or greater, or about 110,000 Da or greater. In specific embodiments, the gelatin can have an average molecular mass of about 75,000 Da to about 250,000 Da, about 90,000 Da to about 225,000 Da, about 100,000 Da to about 200,000 Da, or about 120,000 Da to about 180,000 Da. In specific embodiments, a gelatin having a molecular mass of about 140,000 Da to about 160,000 Da can be particularly useful.

Molecular mass can be expressed as a weight average molecular mass (M_{w}) or a number average molecular mass (Mₙ). Both expressions are based upon the characterization of macromolecular solute containing solution as having an average number of molecules (nᵢ) and a molar mass for each molecule (Mᵢ). Accordingly, number average molecular mass is defined by formula 1 below.

$${M}_{n} = \frac{\sum {n}_{i} {M}_{i}}{\sum {n}_{i}} \qquad ( 1 )$$

Weight average molecular mass (also known as molecular mass average) is directly measurable using light scattering methods and is defined by formula 2 below.

$${M}_{w} = \frac{\sum {n}_{i} {M}_{i}^{2}}{\sum {n}_{i} {M}_{i}} \qquad ( 2 )$$

Molecular mass can also be expressed as a Z-average molar mass (M_{z}), wherein the calculation places greater emphasis on molecules with large molar masses. Z-average molar mass is defined by formula 3 below.

$${M}_{z} = \frac{\sum {n}_{i} {M}_{i}^{3}}{\sum {n}_{i} {M}_{i}^{2}} \qquad ( 3 )$$

Unless otherwise noted, molecular mass is expressed herein as weight average molecular mass.

The gelatin used according to the invention can be derived from a variety of useful sources, such as that obtained by at least partially hydrolyzing collagen that is derived from animal skin, connective tissue, or bones. Alternatively, the gelatin can be from in vitro synthesis, such as from cell culture, can be human-derived, or can be synthetic. Type A gelatin (i.e., derived from an acid-treated precursor) or Type B gelatin (i.e., derived from an alkali-treated precursor) can be used. Moreover, the gelatin can be derived using chemical hydrolysis and/or thermal hydrolysis to denature the collagen.

The polymeric carbohydrate of the composition can be a natural carbohydrate, such as a glycosaminoglycan or can be a synthetic carbohydrate. To have the desired properties to form a solid or semi-solid gel matrix at the desired temperatures with gelatin, a carbohydrate (e.g., dextran) having a molecular mass of approximately 10,000 to 1,000,000 Da can be preferred. Carbohydrates with a molecular mass at the higher end of the range can provide greater matrix physical properties and stability.

The polymeric carbohydrate can encompass a variety of polysaccharides, such as glycosaminoglycans or mucopolysaccharides, and synthetic carbohydrates. Specific, non-limiting examples of polymeric carbohydrates that can be used include agarose, alginate, amylopectin, amylose, carrageenan, cellulose, chitin, chitosan, chondroitin sulfate, dermatan sulfate, dextran, dextran sulfate, glycogen, heparan, heparan sulfate, heparin, hyaluronic acid, keratan sulfate, and starch.

The stoichiometry of the gelatin and the polymeric carbohydrate preferably can be formulated in a desirable range due the complementary ionic charges of the two polymeric components at physiological pH. Since the desired physical and potential biological properties are derived from the polymeric components (gelatin plus polymeric carbohydrate), the preferred stoichiometry range is a minimum of 60% of the polymeric weight comprising gelatin to provide suitable properties as described in the examples. Thus, the gelatin component can comprise about 60% or greater by weight of the combination of the gelatin and the polymeric carbohydrate. In further embodiments, the gelatin component can comprise about 62% or greater, about 65% or greater, about 67% or greater, or about 70% or greater by weight of the combination of the gelatin and the polymeric carbohydrate. In further embodiments, the gelatin can comprise about 60% by weight to about 95% by weight, about 65% by weight to about 90% by weight, or about 70% by weight to about 85% by weight of the combination of the gelatin and the polymeric carbohydrate in the overall composition.

As already noted above, the overall combination of materials forming the present composition, including the aqueous component, can provide for the formation of a material that has a relatively narrow phase change temperature range. Specifically, the interaction of the polymeric materials and the additional components of the composition can form a phase-controllable hydrogel matrix that is solid or semi-solid at temperatures approximately equal to the average skin temperature or below and that can be transitioned to a flowable liquid composition by heating to a temperature that is slightly greater than average skin temperature. In other words, the presently disclosed compositions can provide surprisingly narrow phase control. Specifically, the present compositions can be tailored to have a very narrow and specific transition temperature range such that a difference of, for example, only about 0.5° C. to about 3° C., about 0.8° C. to about 2.5° C., or about 1° C. to about 2° C., can be effective to cause the phase transition from a flowable, injectable liquid to a solid or semi-solid (such as after injection into the dermal tissue), or vice versa. In certain embodiments, the presently disclosed hydrated composition can be a flowable liquid at a temperature of 35° C. or greater, as determined by differential scanning calorimetry (DSC). More particularly, the presently disclosed composition can be a flowable liquid at a temperature of 35° C. or greater, about 36° C. (+/−0.5° C.) or greater, or about 37° C. (+/−0.5° C.) or greater. In other embodiments, the presently disclosed composition can be in a solid or semi-solid gel matrix at a temperature that is less than 35° C.

The total polymer concentration of gelatin and polymeric carbohydrate can range from approximately 50 mg/mL to approximately 400 mg/mL, approximately 75 mg/mL to approximately 350 mg/mL, or approximately 100 mg/mL to approximately 300 mg/mL. The composition can be titrated to near physiological pH to allow the ionic interactions of the two polymeric components to form a gel. Buffers can be utilized to promote the appropriate pH, in the range of about 6 to about 8. Other components such as physiological salts and amino acids can be incorporated into the composition to provide a supportive environment for cells involved in wound healing. Exemplary amino acids that can be used include Glutamic acid, Arginine, Lysine, Cysteine, and Alanyl-Glutamine. A specific isomer, such as the L-isomer, of the amino acids can be used. Exemplary useful salts include edatate disodium and metal salts, such as zinc salts (e.g., zinc sulfate), calcium salts, magnesium salts, sodium salts, and potassium salts.

The compositions of this disclosure are particularly useful in that when they are heated to temperatures that are slightly above average human dermal temperature (i.e., 35° C. or greater), the compositions shift from being a solid or semi-solid material into a flowable liquid. Preferably, the flowable liquid composition exhibits suitable physical characteristics to allow for injection of the composition into the dermal and subdermal tissues, such as those surrounding a dermal wound. In specific embodiments, the physical characteristics are such that the flowable liquid composition is adapted for injection through a relatively small gauge needle. For example, the flowable liquid composition can be adapted to injection through a needle or the like having a nominal inner diameter of about 0.2 mm or greater, about 0.25 mm or greater, or about 0.3 mm or greater. The flowable liquid composition further can be adapted to injection through a needle or the like having a nominal inner diameter of about 0.2 mm to about 0.4 mm or about 0.25 mm to about 0.35 mm. Examples of useful needle gauges include 18 gauge through 27 gauge needles, 23 gauge through 27 gauge needles, or 23 gauge through 25 gauge needles. Since relatively small gauge needles are required to inject a composition into a relatively small target, such as that presented by dermal tissues, it is necessary for injectable compositions to exhibit sufficiently low viscosities. Also, injectable compositions must limit colloidal or insoluble particles to those in a size range that is less than the nominal inner diameter of the injection needle. Thus, it can be beneficial for colloidal or insoluble particles to have an average size that is about one-fifth or less, about one-sixth or less, or about one-eighth or less than the nominal inner diameter of the injection needle. More specifically, the colloidal or insoluble particles can have an average size of about one-fifth to about one-tenth the nominal inner diameter of the injection needle. Such sizing can allow for injection of the composition without needle clogging.

Total solids content similarly can adversely affect flowability and injectability of the present composition. Preferably, when in a flowable, liquid form, it can be desirable for the composition to have a total solids content below a certain threshold, particularly to provide a viscosity suitable for injection through a small gauge needle. For example, it can be beneficial for total solids content to be about 0.5 g/mL or less, about 0.4 g/mL or less, about 0.3 g/mL or less, about 0.2 g/mL or less, or about 0.1 g/mL or less. In further embodiments, the total solids content of the flowable, injectable liquid composition can be about 0.01 g/mL to about 0.5 g/mL, about 0.025 g/mL to about 0.45 g/mL, about 0.05 g/mL to about 0.4 g/mL, or about 0.1 g/mL to about 0.3 g/mL.

Still further to the above, the viscosity of the flowable, liquid composition according to the present disclosure can be relevant to injectability of the material—e.g., through a small gauge needle. More specifically, a significantly high viscosity can limit the ability to inject the composition through with a manual syringe. Preferably, when in a flowable, liquid form, it can be desirable for the composition to have a viscosity of about 2 Pa-s or less, about 1.5 Pa-s or less, about 1.25 Pa-s or less, about 1.1 Pa-s or less, about 1 Pa-s or less, about 0.75 Pa-s or less, about 0.5 Pa-s or less, or about 0.25 Pa-s or less. In further embodiments, a flowable, liquid composition can have a viscosity of about 0.01 Pa-s to about 2 Pa-s, about 0.025 Pa-s to about 1.75 Pa-s, about 0.05 Pa-s to about 1.5 Pa-s, about 0.1 Pa-s to about 1.25 Pa-s, or about 0.2 Pa-s to about 1.1 Pa-s. One method for evaluating viscosity is described below in Example 4, and viscosity values can be referenced to a test temperature range of about 39° C.

The flowability of the hydrated, liquid composition can be characterized in relation to the pressure and flow rate of the composition through a needle. For example, flow rate can be evaluated through use of known fluid dispensing systems, such as those available from Nordson/EFD. In a specific test, 1 mL of a test fluid can be placed in a 1 mL syringe and a syringe plunger force of 5 Newtons (N) can be applied to force the fluid out of the syringe through a 16 mm (⅝ inch) long 25 gauge needle (0.26 mm nominal inner diameter). Such testing is in accord with ISO 7886-1, but other testing conditions also can be used. When the above testing conditions are used, a liquid, hydrated composition according to the present disclosure can have an average flow rate of about 5 μL/s or greater, about 10 μL/s or greater, about 15 μL/s or greater, about 25 μL/s or greater, about 75 μL/s or greater, about 100 μL/s or greater, or about 200 μL/s or greater. Average flow rate particularly can be about 10 μL/s to about 400 μL/s, about 12 μL/s to about 350 μL/s, or about 15 μL/s to about 300 μL/s. The above values similarly can apply to flowability through any gauge needle otherwise described herein. Further, such values for flowability can be referenced to a test temperature range of about 39° C.

When in the solid or semi-solid state, the compositions of the present disclosure can be characterized by the compressive strength of the material. Mechanical testing to evaluate mechanical strength is particularly described below in Example 6, and such testing can be carried out using materials and methods available in the industry. In certain embodiments, a solid or semi-solid composition according to the present disclosure can exhibit a compressive modulus of about 15 kPa or greater, about 20 kPa or greater, or about 25 kPa or greater at ambient conditions. In other embodiments, compressive modulus can be about 15 kPa to about 50 kPa, about 20 kPa to about 45 kPa, or about 20 kPa to about 40 kPa at ambient conditions. In certain further embodiments, the compressive modulus can be characterized as at least about 34 kPa, at least about 35 kPa, or at least about 36 kPa at ambient conditions. In some embodiments, the manufacturing process according to the invention can produce compositions of the invention with relatively high levels of compressive modulus, which may enhance performance of such materials in certain wound treatments. One method for evaluating compressive modulus is discussed in Example 6.

When in the solid or semi-solid state, the compositions of the present disclosure can also be characterized by the dissolution properties of the material. Testing to evaluate dissolution of the materials of the invention is particularly described below in Example 6, and such testing can be carried out using materials and methods available in the industry. In certain embodiments, the composition of the invention formed in disks (sized 8 mm in diameter and 1.5 mm in height) can exhibit complete dissolution, according to visual inspection in phosphate buffered saline at 34° C., in greater than about 45 minutes, greater than about 46 minutes, greater than about 47 minutes, or greater than about 48 minutes. Greater resistance to dissolution generally correlates to enhanced residence time when implanted at a wound site, which may enhance the wound healing function of the compositions of the invention.

The compositions of the present disclosure can be particularly beneficial in relation to the preserved activity of specific components thereof. As discussed above, the process of denaturing collagen (e.g., the chemical processing) can degrade the fibronectin and polymeric carbohydrate binding properties of the resultant gelatin. This can in part relate to the degradation of the alpha chains of the collagen. The compositions of the present disclosure, however, in part because of the preservation of the higher molecular mass collagen alpha chains, can exhibit high fibronectin binding activity (FBA), particularly in comparison to other compositions comprising denatured collagen. In certain embodiments, compositions according to the present disclosure can exhibit a FBA of about 3 nmol/mg or greater, about 4 nmol/mg or greater, or about 5 nmol/mg or greater. Such activity can be measured as the concentration of fibronectin binding sites on the gelatin normalized to the concentration of the gelatin in the composition. One method for measuring fibronectin binding activity is described in Example 5.

As further discussed below, the compositions of the present disclosure particularly can be used in the treatment of the dermal or subdermal tissue of mammals, particularly humans. Generally, the flowable compositions can be injected into an area of the dermal or subdermal tissue, such as in the area of a new, healing, or healed wound. As such, the compositions can be characterized in relation to the residence time of the intact composition within the dermal or subdermal tissue. Such residence time of the composition can also be referred to as the biodegradability time of the composition—i.e., the amount of time from placement until the materials of the composition are degraded or bioabsorbed by the surrounding tissue and physiological processes. In certain embodiments, the residence time of a composition according to the present disclosure in the dermal or subdermal tissue of a mammal, such as a human, can be about 3 days or greater, about 4 days or greater, about 5 days or greater, about 6 days or greater, or about 7 days or greater. In other embodiments, the residence time can be about 3 days to about 60 days, about 4 days to about 45 days, about 5 days to about 40 or about 6 days to about 35 days.

In preparation of the composition of the present disclosure, the polymeric components can be mixed together with sufficient water (or other solvent) to allow the polymers to interact as discussed herein to form a hydrogel matrix. The polymers can be added to a mixing vessel dry and then hydrated together, or separate solutions of the individual polymers can be prepared (e.g., a stock solution, which can be provided at a variety of concentrations for dilution to form the hydrogel composition) and then be mixed together. Buffers or titrating agents can be added to adjust the pH and ionic strength. The combination of components can then be mixed with heating (e.g., to a temperature greater than the liquid transition temperatures discussed herein) to form a uniform hydrogel composition. Such mode of preparation can achieve a hydrogel matrix that is suitable for use in certain applications where therapeutic benefit relies upon the nature of the constituent parts of the composition but not upon the overall structure of the hydrogel itself. In particular, it has been found according to the present disclosure that formation of the hydrogel without utilizing the temperature and mixing specifications and the salt concentration ranges discussed herein can result in a thermoreversible material capable of existing in a solid or semi-solid state but which undergoes phase separation at physiological conditions. Specifically, the gelatin and polymeric carbohydrate components can significantly disassociate such that the hydrogel structure breaks down, liquid components are rapidly resorbed by surrounding tissue, and the separate polymers persist for a time providing a scaffold-like structure. It has further been found according to the present disclosure that while such bio-polymer scaffold can provide some therapeutic benefit, it is inadequate to provide for desirable healing of dermal wounds, particularly acute dermal wounds, and specifically with reduced scarring and reduction or elimination of keloid formation. Such ends can be achieved, however, according to the present disclosure though utilization of a hydrogel composition that is prepared according to the presently defined specifications. In particular, it has been found that the means of preparation of the hydrogel composition has an unexpected effect on the phase stability of the hydrogel under dermal conditions and, as such, the hydrogel compositions of the present disclosure can be further defined by a hydrogel stability factor that can be directly correlated to the method of preparation of the composition.

In certain embodiments, a hydrogel stability factor can be calculated though ultrasonic evaluation of a hydrogel. Any ultrasonic spectrometer effective for non-destructive analysis of hydrogel properties using high-resolution measurements of the velocity and attenuation of acoustical waves propagating through samples at high ultrasonic frequencies may be used. As described in Example 13, an HR-US 102 ultrasonic spectrometer (available from Sonas Technologies, Ltd., Dublin, Ireland) may be used. The ultrasonic velocity provides information on the high frequency elasticity of the hydrogel (or other test medium), which is extremely sensitive to intermolecular interactions, hydration, micro-elasticity, cross-linking, and internal structure and composition of a sample. The ultrasonic attenuation is determined by the energy losses in the ultrasonic wave propagating through the sample and is proportional to the high frequency viscosity. Such evaluation is useful to provide information on microstructural organization of samples and its evolution (e.g., particle size, aggregation, and coalescence). Ultrasonic testing has thus been found according to the present disclosure to be a reliable means for evaluating the dermal stability of a thermoreversible hydrogel and correlating such dermal stability to the method of preparation of the hydrogel composition.

One preferred embodiment for preparation of a composition with a desirable hydrogel stability is described in Example 1. In various embodiments, a portion of the non-polymer components of the composition can be mixed into an aqueous medium (Medium 199 in Example 1) and allowed to equilibrate. The mixing of the components to form the liquid hydrogel composition preferably is carried out at a first temperature adapted to increase the availability of binding sites on the gelatin for interaction with the polymeric carbohydrate. In certain embodiments, the first mixing temperature can be a temperature of about 45° C. or greater or about 50° C. or greater (e.g., about 45° C. to about 80° C., about 46° C. to about 70° C., about 48° C. to about 65° C., or about 50° C. to about 60° C.). Thereafter, the polymeric carbohydrate and the gelatin can be added to the solution sequentially or in combination (including dry forms and stock polymer solutions). Once the polymers are in solution, the pH of the hydrogel composition can be adjusted to the desired range. After equilibration from the pH adjustment, the remaining non-polymer components can be stirred into the liquid hydrogel composition. The hydrogel processing during which all hydrogel components are hydrated or otherwise formed into liquid solution preferably is carried out with constant mixing or like agitation that substantially maintains the hydrogel components in solution. For example, a stir bar or like mixing device may be used. In some embodiments, formation of the liquid hydrogel composition can be carried out with constant mixing at the first temperature for a time of about 10 minutes or greater, about 30 minutes or greater, about 60 minutes or greater, or about 90 minutes or greater (e.g., about 20 minutes to about 300 minutes, about 30 minutes to about 240 minutes, or about 45 minutes to about 180 minutes).

It has been found according to the present disclosure that hydrogel stability is directly affected by the temperature conditions of the preparation method. While mixing at the temperature defined above is useful for solubilization of the composition materials and thus initial formation of the liquid hydrogel composition, it is beneficial for the composition to be cooled to a holding temperature that is less than the first temperature before cessation of mixing. The holding temperature can be defined in relation to the first temperature and/or in relation to the gelation temperature of the hydrogel composition. In some embodiments, the holding temperature can be at least about 2° C., at least about 5° C., or at least about 8° C. less than the first temperature. In further embodiments, the holding temperature can be within about 10° C., within about 7° C., within about 5° C., or within about 2° C. of the gelation temperature of the hydrogel composition. The gelation temperature is understood to be the temperature at which the liquid hydrogel composition transitions into a solid or semi-solid hydrogel composition. In certain embodiments, the holding temperature can be less than about 45° C. or less than about 42° C. (e.g., about 36° C. to about 44° C., about 37° C. to about 43° C., or about 38° C. to about 42° C.). For example, the equilibrated, liquid hydrogel composition at about 50° C. can be pumped from the mixing vessel into a holding vessel at about 40° C. (during which filter sterilization may be carried out). Once the composition components are sufficiently solubilized as described above so as to form the liquid hydrogel composition, it can be cooled to the holding temperature using any cooling means. In preferred embodiments, the holding vessel can be actively cooled so that the liquid hydrogel composition entering the holding vessel is cooled to the holding temperature. Cooling also may be carried out during filtering or through other means. In some embodiments, it may be preferable to cool the formed, liquid hydrogel to the holding temperature in a defined time period, such as, for example, in a time of less than about 8 hours, less than about 4 hours, less than about 2 hours, or less than about 1 hour. It is particularly preferred for the liquid hydrogel composition to be subject to constant mixing during cooling to the holding temperature. It also can be preferred for the liquid hydrogel composition to be subject to constant mixing for the entirety of the time that the liquid hydrogel composition is held at the holding temperature. Application of constant mixing prior to cooling of the liquid composition to a gelation temperature can be characterized as maintaining the liquid hydrogel composition as a homogeneous liquid polymeric solution. Premature cessation of mixing can cause the liquid polymeric solution to be non-homogeneous in that phase separation of the gelatin and polymeric carbohydrate occurs, and the polymeric components are not homogeneously dispersed throughout the liquid composition so that the solid or semi-solid hydrogel that form below the gelation temperature achieves necessary bonding between the polymeric components.

The liquid hydrogel composition at the holding temperature can be dispensed into the desired packaging or storage containers or maintained in a bulk composition. Thereafter, the liquid hydrogel composition can be further cooled to a storage temperature that is less than the gelation temperature of the composition (i.e., is at a temperature wherein the composition is in a solid or semi-solid state). In some embodiments, cooling to the holding temperature can be carried out using a first method, and further cooling to the gelation temperature can be carried out using a second, different method. For example, the first method can include mixing, and the second method can be in the absence of mixing. As a further example, the first method can comprise cooling to the desired temperature in a first amount of time, and the second method can comprise cooling to the gelation temperature in a second, different amount of time. In some embodiments, cooling of the hydrogel composition can be substantially stopped at the holding temperature for a minimum time. Thus, it is evident that the step of cooling to the holding temperature and the step of cooling to the gelation temperature are separate and distinct steps, and a suitable hydrogel according to the present disclosure is not achieved when cooling directly from the first temperature to the gelation temperature without first cooling to the holding temperature. Since the holding temperature is substantially close to the gelation temperature, as discussed above, cooling to the storage temperature can be carried out in the absence of mixing without undesirable effects on the composition—i.e., without substantial loss in hydrogel polymeric homogeneity. The solid or semi-solid hydrogel composition that forms as the cooling transitions the composition through the gelation stage can be characterized as being in a stabilized form in that the solid or semi-solid gel forms sufficiently rapidly to avoid substantial phase separation or structural rearrangement of the homogeneously mixed liquid composition. The solid or semi-solid hydrogel composition can be stored at a temperature substantially below the gelation temperature of the composition, such as under refrigeration, particularly at a temperature of less than about 20° C., less than about 15° C., or less than about 10° C. (e.g., at a temperature of about 1° C. to about 12° C., about 2° C. to about 10° C., or about 2° C. to about 8° C.). Preferably, the time between cessation of mixing and gelation to form the solid or semi-solid hydrogel composition is less than about 2 hours, less than about 1 hour, or less than about 30 minutes.

The nature of the medium (e.g., water) used in preparing the hydrogel composition also can significantly affect the hydrogel stability. Preferably, the hydrogel medium can be deionized water and can be supplemented with one or more buffers and/or preservatives. It further is preferable, however, for the hydrogel medium to avoid excessive salt concentrations, particularly in relation to specific ions. In various embodiments, the aqueous medium used in forming the liquid hydrogel composition can be defined by a osmolality below a specified concentration. For example, the aqueous medium can be less than about 400 mOsm/kg in total salt concentration. In further embodiments, the osmolality of the aqueous medium can be less than about 350 mOsm/kg, less than about 325 mOsm/kg, or less than about 300 mOsm/kg. In particular, the osmolality of the aqueous medium can be about 10 mOsm/kg to about 400 mOsm/kg, about 25 mOsm/kg to about 375 mOsm/kg, or about 50 mOsm/kg to about 350 mOsm/kg. Although not wishing to be bound by theory, it is believed that the above osmolality conditions sufficiently mimic the nature of the extracellular matrix in mammalian dermal tissue so as to affect the ability of the solid or semi-solid hydrogel composition to maintain its gelled structure for a sufficient residence time to achieve the desired treatment effect as discussed herein. Thus, in certain embodiments, the hydrogel composition of the present disclosure can be defined as having an osmolality that exceeds the osmolality of the extracellular matrix of the treated tissue by no greater than about 20%, no greater than about 18%, or no greater than about 15%.

In further embodiments, the nature of the aqueous medium also can be defined in relation the phosphate ion concentration and/or the carbonate ion concentration. For example, a phosphate buffered saline (PBS) has been found to provide unacceptably high phosphate concentrations to achieve a stable hydrogel according to the present disclosure. This is illustrated in the appended Example 2. Preferably, an aqueous medium used according to the present disclosure has a phosphate ion concentration that is no greater about 25 mM, no greater than about 20 mM, or no greater than about 15 mM. Similarly, an aqueous medium used according to the present disclosure has a carbonate ion concentration that is no greater about 25 mM, no greater than about 20 mM, or no greater than about 15 mM. On the other hand, Medium 199, as used in appended Example 1, has been found to be a useful hydrogel medium with sufficiently low phosphate ion concentrations to provide for formation of a highly stable hydrogel composition.

Although the above processing parameters for the preparation of a stable hydrogel composition are recited separately, it is understood that methods according to the present disclosure can be defined by one of the noted processing parameters or by any combination of the noted processing parameters. Moreover, the individual processing parameters may be carried out in accordance with any aspect of the further description thereof provided herein. For example, a method for preparing a useful stable hydrogel composition comprising gelatin and a polymeric carbohydrate can be defined in relation to any of the following parameters:

mixing at a first temperature and cooling to a holding temperature as described herein;

maintaining mixing while cooling as described herein;

utilizing a medium with a defined phosphate concentration as described herein;

utilizing a medium with a defined carbonate concentration as described herein;

mixing at a first temperature and cooling to a holding temperature as well as maintaining mixing while cooling as described herein;

mixing at a first temperature and cooling to a holding temperature as well as utilizing a medium with a defined phosphate concentration as described herein;

mixing at a first temperature and cooling to a holding temperature as well as utilizing a medium with a defined carbonate concentration as described herein;

mixing at a first temperature and cooling to a holding temperature as well as maintaining mixing while cooling and utilizing a medium with a defined phosphate concentration as described herein;

mixing at a first temperature and cooling to a holding temperature as well as maintaining mixing while cooling and utilizing a medium with a defined carbonate concentration as described herein; and

mixing at a first temperature and cooling to a holding temperature as well as maintaining mixing while cooling, utilizing a medium with a defined phosphate concentration, and utilizing a medium with a defined carbonate concentration as described herein.

In light of the foregoing, a hydrogel composition useful according to the present disclosure can be defined as a hydrogel with a compositional makeup that was formed under processing conditions to provide a specific hydrogel stability factor. This hydrogel stability factor then can be directly correlated to the ability of the hydrogel composition for effective treatment of acute dermal wounds, including scar revision. Testing described in the appended Examples has shown that even when the same two polymers are used in the same amount to form a hydrogel matrix that differs mainly only in the solvent medium used (e.g., Medium 199 versus a high phosphate PBS in Examples 1 and 2), the process by which the hydrogel composition is prepared can significantly alter the hydrogel stability of the composition. Specifically, a hydrogel composition with a greater stability factor according to the present disclosure is believed to persist in dermal tissue for a sufficient duration while maintaining the hydrogel structure arising from the bonding interactions of the hydrogel polymers so as to achieve a level of treatment efficacy (e.g., scar reduction) that significantly and unexpectedly exceeds that of a like hydrogel with a lesser stability factor. Specifically, the hydrogel with a lesser stability factor can undergo phase separation and hydrogel rearrangement after an unacceptably short dermal residence time so that the necessary wound healing mechanisms that are facilitated by the hydrogel structure of the composition cannot proceed in a manner that reduces scarring, particularly keloid formation. This difference is surprising in that the stabilizing effect of the temperature, mixing, and salt concentration parameters described herein on formation of a stabilized, solid or semi-solid hydrogel composition carries over once the hydrogel has been liquefied for injection and re-formed into the solid or semi-solid form in dermal tissue.

Hydrogel compositions that exhibit a useful stability for acute dermal wound treatment according to the present disclosure can be confirmed as such through ultrasonic testing as described herein. A hydrogel stability factor as used herein can be defined as the percentage change in ultrasonic attenuation (U_{A}) over minimum defined time of ultrasonic testing at a defined frequency or frequency range and a defined testing temperature. For example, a U_{A} hydrogel stability factor of 0.5 corresponds to a change in ultrasonic attenuation of less than 50% over the defined time interval.

In an example embodiment, a hydrogel composition useful according to the present disclosure can exhibit a U_{A} hydrogel stability factor of 0.4 for at least 500 minutes when tested at a temperature of 35° C. and a frequency of 2-8 MHz. Thus, the ultrasonic attenuation of the hydrogel will change by less than 40% for a time of at least 500 minutes under the stated test conditions. In another example embodiment, a hydrogel composition useful according to the present disclosure can exhibit a U_{A} hydrogel stability factor of 0.5 for at least 600 minutes when tested at a temperature of 35° C. and a frequency of 2-8 MHz. Thus, the ultrasonic attenuation of the hydrogel will change by less than 50% for a time of at least 600 minutes under the stated test conditions. A lower hydrogel stability factor corresponds to a more stable hydrogel that resists phase separation and structural rearrangement for a sufficient dermal residence time to provide effective scar reduction treatment.

In one embodiment, a hydrogel composition useful according to the present disclosure can have a U_{A} hydrogel stability factor of 0.3 for at least 500 minutes when tested at a temperature of 35° C. and a frequency of 2.7 MHz. In another embodiment, a useful hydrogel composition can have a U_{A} hydrogel stability factor of 0.4 for at least 600 minutes when tested at a temperature of 35° C. and a frequency of 2.7 MHz. In another embodiment, a useful hydrogel composition can have a U_{A} hydrogel stability factor of 0.2 for at least 500 minutes when tested at a temperature of 35° C. and a frequency of 5.1 MHz. In another embodiment, a useful hydrogel composition can have a U_{A} hydrogel stability factor of 0.3 for at least 600 minutes when tested at a temperature of 35° C. and a frequency of 5.1 MHz. In another embodiment, a useful hydrogel composition can have a U_{A} hydrogel stability factor of 0.35 for at least 500 minutes when tested at a temperature of 35° C. and a frequency of 7.8 MHz. In another embodiment, a useful hydrogel composition can have a U_{A} hydrogel stability factor of 0.5 for at least 600 minutes when tested at a temperature of 35° C. and a frequency of 7.8 MHz.

To further promote stability of the resultant gel matrix, it can be desirable to concentrate the portions of the polymers that contribute to the stability of the final composition in-situ. Methods can be used in production of the composition to remove the most soluble portions of each polymer or of the mixed composition to concentrate the portions of each polymer with the desired properties. One method to concentrate the desired polymer can be to slowly solubilize the polymers together while heating and to decant or remove the most soluble liquid to concentrate the remaining polymer mixture. Another method to concentrate the composition can be to wash each polymer or the mixture of the polymers while in a partially soluble condition, such as due to exposure with water/alcohol mixture or with high ionic strength conditions such as with NaCl solutions. Such gel washing techniques can result in a composition that retains the less soluble components and thus enhances physical properties of the composition and improves persistence in the treatment site. It is particularly beneficial to concentrate the composition while the polymers are mixed in the desired pH range to allow ionic interactions between the polymers and fractionation to remove the more soluble portions of the polymer composition.

Bulk fractionation methods known in the art can be used to concentrate the polymers as discussed above. For example, size exclusion chromatography (e.g., gel filtration chromatography or gel permeation chromatography) or Baker-Williams fractionation. Further fractionation methods that can be useful in preparation of the present compositions can be found in Francuskiewicz, Polymer Fractionation, Springer-Verlag, 1994, the disclosure of which is incorporated herein by reference in its entirety. Such fractionated compositions are expected to have greater stability and improved physical properties when in a solid or semi-solid state.

The composition of the disclosure can also include other active agents to either facilitate its use, or to enhance its beneficial effect on a site in need of treatment. Such active agents can include hemostatic agents (including collagen or thrombin), antimicrobial agents (including antibiotics), bacteriocidal or bacteriostatic agents, growth factors (including epidermal growth factor, fibroblast growth factor, platelet derived growth factor, or insulin-like growth factor), or anti-inflammatory agents (such as corticosteroids or non-steroidal anti-inflammatory agents).

To facilitate shipping and storage at ambient conditions, the composition can be lyophilized to a dry state and reconstituted or rehydrated prior to use. The lyophilized composition can provide improved stability and allow room temperature storage prior to use. Exemplary lyophilization methods that can be used according to the invention are disclosed in the following: U.S. Pat. No. 5,192,743; U.S. Pat. No. 7,666,413; U.S. Pat. No. 7,695,736; and US Patent Publication No. 2008/0145404. The disclosures of all of the preceding documents are incorporated herein by reference in their entireties. Due to the unique use of the composition, the formulation for lyophilization can be tailored to enable rapid reconstitution of the product. For example, reconstitution can be substantially complete within a time of about 30 seconds to about 90 minutes. In specific embodiments, the time to substantially complete reconstitution can be about 60 minutes or less, about 30 minutes or less, about 20 minutes or less, about 15 minutes or less, or about 10 minutes or less. The time for reconstitution particularly can be in the range of about 30 seconds to about 90 minutes, about 60 seconds to about 60 minutes, about 2 minutes to about 30 minutes, about 3 minutes to about 20 minutes, or about 4 minutes to about 15 minutes. Heating can be applied during reconstitution and/or the liquid used for reconstitution can be pre-heated to a specific temperature. For example heating to a temperature of 35° C. or greater can be beneficial. Preferably the reconstitution comprises the addition of an aqueous solution to the lyophilized composition and mixing without the use of mixing methods that would require opening of the container containing the composition and aseptic handling which would complicate use by medical personnel. Means to facilitate reconstitution can include formulation of the composition in a dilute concentration to result in a lower density and more porous dry material. The dried material subsequently can be rehydrated with less fluid than was required to prepare the composition for lyophilization.

The preferential reconstitution times of the present compositions can be facilitated through the use of further components. For example, surfactants can be utilized to minimize hydrophobic interactions within the lyophilized composition that may limit water penetration necessary for reconstitution. Non-limiting examples of suitable surfactants include anionic surfactants (e.g., fatty acids, salts of fatty acids, and alkyl sulfates including sodium lauryl sulfate), cationic surfactants (e.g., ceramide), non-ionic surfactants (e.g., polyol esters, polyoxyethylene esters, and polysorbates, including polysorbate 20 and polysorbate 80), and amphoteric or zwitterionic surfactants. As further examples, hygroscopic excipients also can be used, such as polyethylene glycol, polyols (e.g., glycerin), cyclodextrins, sodium chloride, potassium chloride, magnesium chloride, calcium chloride, and zinc chloride. Likewise, a variety of bulking agents can be utilized in the preparation of the initial hydrogel matrix composition and/or can be added during reconstitution of the lyophilized composition. Although not wishing to be bound by theory, it is believed that the addition of such bulking agents can lead to a lyophilized product that can be more easily penetrated by the water or other solvent used for reconstitution. Exemplary bulking agents that can be used include monosaccharides and disaccharides (for example, dextrose, sucrose, or trehalose), and sugar alcohols, such as mannitol.

As noted above, buffer solutions can be utilized in the preparation of the hydrogel matrix compositions, and appropriate buffers can be added during formation of the initial composition and/or reconstitution of the lyophilized composition. Exemplary buffers that can be used include tris(hydroxymethyl)aminomethane, citrate, glycine, and 4-(2-hydroxyethyl)-1-piperazineethanesulfonic acid. Further, preservatives can be added and can be useful to preserve the composition during dehydration or lyophilization.

In some embodiments, certain surfactants, hygroscopic excipients, and bulking agents (or combinations thereof) can be useful to improve the lyophilization conditions, facilitate preservation after lyophilization, and/or improve the properties of the reconstituted material. Preferred additives for reconstitution can be those that do not interfere with the ability of the composition to form a flowable and injectable fluid at elevated temperature and a solid or semi-solid hydrogel matrix at skin (i.e., dermal and/or subdermal) temperatures, particularly over a narrow, controlled phase change temperature.

In various embodiments, a combination of reconstitution additives can be used to provide a hydrogel that is reconstituted from a lyophilized form, that reconstitutes in a desirably rapid time, and that reconstitutes to a fluid form with desirable injectability characteristics. The reconstitution additives may be included with the hydrogel prior to lyophilization such that reconstitution additive residues remain in the lyophilized composition and provide the desired reconstitution effects, as discussed herein. The reconstitution additives may be present only in the reconstitution fluid that is used to reconstitute the lyophilized composition. The reconstitution additives may be included with the hydrogel prior to lyophilization such that reconstitution residues are present in the lyophilized composition and may also be included with the reconstitution fluid. Specifically, the additives included before reconstitution may be different from the additives included with the reconstitution fluid, or the pre-lyophilization additives and the reconstitution additives included in the reconstitution fluid may have one or more materials in common.

The amount of reconstitution additives present in the reconstituted composition can be defined by the concentration of the reconstitution additive material in relation to the reconstitution fluid (either as a direct additive in the reconstitution medium—e.g., deionized water, or as a residue carried into the reconstitution fluid in the lyophilized composition). In some embodiments, a reconstitution fluid can comprise up to about 4%, up to about 3%, up to about 2%, or up to about 1% by weight of any one of a surfactant, a hygroscopic excipient, and a bulking agent. In other embodiments, a reconstitution fluid can comprise about 0.01% to about 4%, about 0.015% to about 3%, about 0.02% to about 2%, or about 0.025% to about 1.5% by weight of any one of a surfactant, a hygroscopic excipient, and a bulking agent. The total reconstitution fluid may comprise up to about 10%, up to about 8%, up to about 6%, or up to about 4% by weight of all combined reconstitution additives. In further embodiments, the total reconstitution fluid may comprise about 0.05% to about 6%, about 0.1% to about 5%, about 0.25% to about 4%, or about 0.5% to about 3% by weight of all combined reconstitution additives. The reconstitution additives may be provided in deionized water.

In certain embodiments, a combination of reconstitution additives can comprise any of the following: i) a surfactant and a hygroscopic excipient, such as a polysorbate and a polyol (e.g., Tween and glycerol) or a polysorbate and a salt (e.g., Tween and NaCl); ii) a surfactant and a bulking agent, such as a polysorbate and a sugar (e.g., Tween and a disaccharide, such as dextrose); iii) a hygroscopic excipient and a bulking agent, such as a polyol and a sugar (e.g., glycerol and a disaccharide, such as dextrose) or a salt and a sugar (e.g., NaCl and a disaccharide, such as dextrose); and iv) a surfactant, a hygroscopic excipient, and a bulking agent, such as a polysorbate, a polyol, and a sugar (e.g., Tween, glycerol, and a disaccharide, such as dextrose) or a polysorbate, a salt, and a sugar (e.g., Tween, NaCl, and a disaccharide, such as dextrose). A surfactant (such as a polysorbate) and a hygroscopic excipient (such as a polyol or a salt) can be present in a ratio of about 1:20 to about 20:1. A surfactant (such as a polysorbate) and a bulking agent (such as a disaccharide, e.g., dextrose) can be present in a ratio of about 1:40 to about 10:1. A hygroscopic excipient (such as a polyol or a salt) and a bulking agent (such as a disaccharide, e.g., dextrose) can be present in a ratio of about 1:10 to about 10:1.

In one embodiment, a reconstitution fluid can comprise about 0.01% to about 4% by weight of a surfactant and about 0.01% to about 4% by weight of a hygroscopic excipient in deionized water. In another embodiment, a reconstitution fluid can comprise about 0.01% to about 4% by weight of a surfactant and about 0.01% to about 4% by weight of a bulking agent in deionized water. In a further embodiment, a reconstitution fluid can comprise about 0.01% to about 4% by weight of a hygroscopic excipient and about 0.01% to about 4% by weight of a bulking agent in deionized water. In yet another embodiment, a reconstitution fluid can comprise about 0.01% to about 4% by weight of a surfactant, about 0.01% to about 4% by weight of a hygroscopic agent, and about 0.01% to about 4% by weight of a bulking agent in deionized water.

The present disclosure also provides various methods of treatment whereby the compositions can be applied to cutaneous wounds, including surgical incisions and excision sites, to improve the healing process of the wound and thus prevent or reduce the occurrence of scarring, including hypertrophic scarring and keloid scarring, and prevent or reduce an effect of scarring. Application of the compositions of the present disclosure can vary depending upon the nature of the treatment site and can include topical application as well as injection, such as into the dermal or subdermal tissue in the proximity of the wound (i.e., along one side of the wound or partially or completely surrounding the wound). When injection is used, the composition particularly can be adapted for injection through a small gauge needle (e.g., such as 23 gauge, 25 gauge, or 27 gauge), as already discussed above. In addition to application of the composition, the methods also can include closing the wound, such as using a closure selected from the group consisting of sutures, staples, glues, and combinations thereof.

In some embodiments, the methods can encompass application to an accidental wound, such as a cut or a burn to the skin, or a chronic wound. In other embodiments, the methods can encompass application to a surgically created wound, such as a surgical incision. Specifically, the surgical wound can be a wound remaining after excision of a pre-existing scar (e.g., a hypertrophic scar, a keloid scar, or a burn-related scar).

In specific embodiments, the disclosed methods can include methods for the revision of a cutaneous keloid or hypertrophic scar (or other types of scars, including burn-related scars). A hypertrophic scar may be characterized as a raised scar arising from overproduction of collagen. A keloid may be characterized as a benign fibrous proliferation resulting in a large hyperplastic mass or soft tissue tumors. Such revision method can comprise excising at least a portion of the scar tissue so as to form an excision site (i.e., a site from which tissue has been surgically removed). The methods further can comprise applying a matrix composition as described herein to the dermal or subdermal tissue in and/or around the excision site. Thus, the composition can be applied directly to the exposed tissue within the excision site and/or the composition can be injected to the surrounding tissue, as described above.

The amount of the composition applied can vary based upon the dimensions of the treatment area. In some embodiments, the hydrated composition can be applied in a volume relative a dimension of the wound. For example, relative volumes can reference the wound margin (i.e., each side of an incision). In some embodiments, total volume of application can be in an amount of about 0.1 mL to about 100 mL, about 0.5 mL to about 75 mL, or about 1 mL to about 50 mL. In other embodiments, the hydrated composition an be applied in a relative volume of about 0.1 mL to about 10 mL per 2.5 cm of the wound margin, about 0.2 mL to about 8 mL per 2.5 cm of the wound margin, about 0.25 mL to about 6 mL per 2.5 cm of the wound margin, or about 0.5 mL to about 4 mL per 2.5 cm of the wound margin. If desired, the methods further can comprise closing the excision site with one or more closures. The closure, for example, can comprise staples, sutures, and glues. In various embodiments, the composition can be injected before and/or after closure.

Scars of significant size can cause pain or discomfort and can be a source of social anxiety or embarrassment. Surgical scar revision according to the present disclosure can be particularly beneficial in that a pre-existing scar can not only be removed, but the recurrence of the scar can be prevented or reduced. It is recognized in the field that patients that have previously been prone to hypertrophic scarring or keloid formation are likely to experience recurrence of scars of a similar size and nature even after revision surgery. Studies have shown that recurrence is seen in at least 50% of revision surgeries, and typically more. The use of the compositions of the present disclosure in combination with surgical revision can greatly reduce this problem. The beneficial effect can particularly be seen in relation to the volume of scar tissue that forms outside of the normal skin boundary layer. Evaluation of scar volume is particularly discussed in Example 12. In specific embodiments, the scar tissue volume present 12 months after revision surgery with application of the present matrix composition relative to the scar tissue volume prior to revision surgery is about 15% or less. In other words, the revision methods described herein can reduce external scar volume by 85% or greater. In further embodiments, the relative external scar volume 12 months after revision surgery can be 10% or less, 5% or less, or 2% or less.

The disclosed use of the composition likewise can prevent or reduce recurrence of keloid formation such that treatment results in no measurable, raised scar tissue at a time of 12 months after treatment. For example, a reduced rate of recurrence at 12 months post-treatment can be 20% or less, 15% or less, 10% or less, or 5% or less of treated patients experiencing no measurable, raised scar tissue.

Treatments and uses of the compositions according to the disclosure further can be characterized in relation to effects of scarring. Thus, not only can the use and application of the compositions prevent or reduce scarring, including hypertrophic scarring and keloid formation, the use and application of the compositions can prevent or reduce an effect of scarring and thus prevent or reduce patient discomfort and/or dissatisfaction associated with any scar tissue formed. For example, effects of scars that can be prevented or reduced include one or more of pain, itching, discoloration of the scar tissue and/or surrounding tissue, abnormal stiffness of the scar tissue or surrounding tissue relative to the subject's normal skin tissue, abnormal thickness of the scar tissue or surrounding tissue relative to the subject's normal skin tissue, and surface irregularity of the scar tissue or surrounding tissue (e.g., roughness and unevenness).

It is believed that the foregoing discussion in combination with the appended examples meets the necessary disclosure requirements for the preparation of a phase-controllable matrix composition that is useful in treating wound sites (chronic, traumatic, or surgical) to reduce or prevent post-surgical scarring, hypertrophic scarring, burn scarring, and/or keloid formation. Further exemplary materials and methods of preparing compositions that can be useful in combination with the present disclosure are provided in the following: U.S. Pat. No. 5,824,331; U.S. Pat. No. 6,231,881; U.S. Pat. No. 6,261,587; U.S. Pat. No. 6,352,707; U.S. Pat. No. 6,713,079; U.S. Pat. No. 6,992,062; U.S. Pat. No. 6,730,315; U.S. Pat. No. 7,303,814; U.S. Pat. No. 7,700,660; U.S. Pat. No. 7,799,767; U.S. Pat. No. 8,053,423; US Patent Publication No. 2008/0145404; US Patent Publication No. 2008/0145404; US Patent Publication No. 2008/0199508; US Patent Publication No. 2009/0123547; and US Patent Publication No. 2009/0124552. The disclosures of all of the preceding documents are incorporated herein by reference in their entireties.

In further embodiments, the present disclosure also provides kits or other articles of manufacture adapted for providing a combination of materials for use in formation of a hydrogel composition and/or use of a hydrogel composition in treatment of a cutaneous wound—e.g., in an acute dermal wound, such as a scar revision treatment or a scar prevention treatment. In certain embodiments, an article according to the present disclosure can comprise a packaging member adapted for holding a plurality of items therein. An exemplary packaging member may be a case, a box, a thermoformed article (e.g., a blister pack or foam pack), or the like.

An article according to the present disclosure further can include a first container and a second container that include two or more materials useful for forming the hydrogel composition. For example, the first container may include a lyophilized composition as otherwise described herein. In an example embodiment, the lyophilized composition may comprise gelatin and a polymeric carbohydrate as well as any residue components from an originally formed hydrogel that was processed to provide the lyophilized composition.

As a further example, the second container may include a reconstitution material. Such reconstitution material may include any material otherwise discussed herein that may be utilized in the reconstitution of a lyophilized hydrogel composition. For example, the reconstitution material may include a reconstitution fluid, particularly an aqueous fluid. The reconstitution material may include one or more additives as described herein that are beneficial for improving the reconstitution of the lyophilized composition, such as surfactants, hygroscopic excipients, and bulking agents. One or more of such additives may be present in the first container as a residue with the lyophilized composition.

The article further may include instructions related to the reconstitution of the lyophilized composition. Such instructions may be particularly directed to a health care provider, particularly a surgeon, and may define the manner of mixing of the contents of the first and second containers so as to achieve a reconstituted hydrogel that exhibits efficacy in dermal wound treatment, particularly scar revision and/or scar prevention or reduction. The instructions may include details for adding an aqueous fluid, such as deionized water, to one or both of the first and second containers, and such deionized water may or may not be included with the respective kit.

The article further may include a connector adapted for one or both of sterile transfer of the contents of the first container into the second container and sterile transfer of the contents of the second container into the first container. In some embodiments, at least one of the first container and the second container can be a syringe. As an example, a luer connector may be included for attaching the first container and the second container to each other or to a further device. In other embodiments, the article may include a needle adapted for attachment to the syringe. As such, the contents of the containers may be combined to form the hydrogel, which then can be directly applied at the dermal wound without the requirement of a mixing apparatus external to the provided article or kit. In some embodiments, a needle sized particularly for dermal treatments to limit tissue trauma may be used. For example the needle may be a 23 gauge through 27 gauge needle.

In use, the contents of the containers in the kit can be combined, such as through transferring an aqueous fluid between the containers to solubilize and hydrate the non-aqueous materials and thus form the hydrogel composition. In an example embodiment, both containers may be a syringe, and the syringes may be connected with a luer connector such that aqueous fluid in one syringe is transferred to the other syringe to effect hydration of the materials therein. The transfer may be repeated back and forth for a time to ensure homogeneous mixing and reconstitution to form the hydrogel composition. The syringe finally containing the reconstituted hydrogel can then be fitted with an appropriate delivery device, such as a needle, to apply the reconstituted hydrogel composition to the dermal wound where treatment is desired.

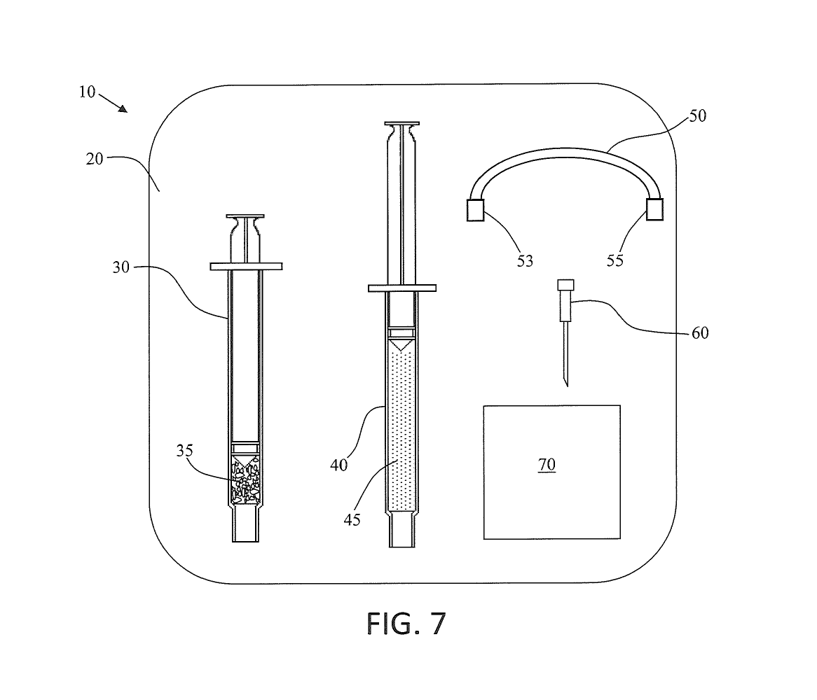

An example embodiment of a kit according to the present disclosure is illustrated in FIG. 7. As seen therein, the kit 10 comprises a package 20, a first container 30, a second container 40, a connector 50 with two connecting ends (53 and 55), a needle 60, and an instruction set 70, which may be written or in digital format. The first container 30 is a syringe that includes a lyophilized hydrogel composition 35. The second container 40 also is a syringe and contains a reconstitution fluid 45, which may include one or more reconstitution additives, as described herein. Further containers may be included. For example, the second container 40 can include the reconstitution fluid 45, and one or more reconstitution additives may be included in a third container.

The present disclosure provides a composition formed of a plurality of components to achieve a hydrogel composition with defined specifications. Although the components and specifications defining the compositions may be separately described, the present disclosure is intended to encompass a variety of combinations of the materials and the achieved specifications thereof. Moreover, the various compositions are each recognized as useful in the treatment of dermal wounds, particularly scar prevention and scar revision, and more particularly treatment or prevention of keloids. Further, the various compositions with the defined specifications and their beneficial uses may arise from the methods of preparation disclosed herein. Provided below are certain exemplary embodiments of the present invention that illustrate the usefulness of the present compositions, methods of manufacture thereof, and methods treatment therewith. Such exemplary embodiments are for purposes of illustration and should not be viewed as limiting the scope of further compositions, defined specifications, methods of manufacture, or methods of treatment that may be encompassed by the further disclosure provided herein.

In one embodiment, the present disclosure provides a stable hydrogel composition comprising gelatin and a polymeric carbohydrate (e.g., dextran) in a combination such that the gelatin comprises about 60% by weight or greater of the total weight of the combination of the gelatin and the polymeric carbohydrate present in the composition and such that the total concentration of the gelatin and the polymeric carbohydrate in the composition is about 50 mg/mL to about 400 mg/mL, and such that the hydrogel composition is a flowable, injectable liquid at a temperature of 35° C. or greater and is a solid or semi-solid gel matrix at lower temperatures. The same stable hydrogel composition may be defined by exhibiting an ultrasonic attenuation (U_{A}) hydrogel stability factor of 0.4 for at least 500 minutes when tested at a temperature of 35° C. and a frequency of 2-8 MHz, more particularly a U_{A} hydrogel stability factor of 0.3 for at least 500 minutes when tested at a temperature of 35° C. and a frequency of 2.7 MHz, a U_{A} hydrogel stability factor of 0.2 for at least 500 minutes when tested at a temperature of 35° C. and a frequency of 5.1 MHz, or a U_{A} hydrogel stability factor of 0.35 for at least 500 minutes when tested at a temperature of 35° C. and a frequency of 7.8 MHz. The same stable hydrogel composition may be defined by a flow rate of about 10 μL/s or greater when forced from a syringe through a ⅝ inch long 25 gauge needle under a syringe plunger pressure of 5 N at a temperature of 35° C. to 39° C. The same stable hydrogel composition may be defined by a viscosity of about 1.5 Pa-s or less at a temperature of 35° C. to 39° C. The same stable hydrogel composition may be defined by having a total osmolality of less than about 400 mOsm/kg, more particularly about 25 mOsm/kg to about 375 mOsm/kg. The same stable hydrogel composition may be defined by having a phosphate ion concentration of no greater than about 20 mM. The same stable hydrogel composition may be defined by having a carbonate ion concentration of no greater than about 20 mM.

Although the above, characteristics of a stable hydrogel composition are recited separately, it is understood that a useful composition according to the present disclosure can be defined by one of the noted characteristics or by any combination of the noted characteristics. Moreover, the individual characteristics may be within any of the value ranges otherwise described herein. For example, a useful stable hydrogel composition comprising gelatin and a polymeric carbohydrate (with the gelatin comprising about 60% by weight or greater of the total weight of the combination of the gelatin and the polymeric carbohydrate present in the composition, with the total concentration of the gelatin and the polymeric carbohydrate in the composition being about 50 mg/mL to about 400 mg/mL, and with the hydrogel composition being a flowable, injectable liquid at a temperature of 35° C. or greater and is a solid or semi-solid gel matrix at lower temperatures) can be characterized in any of the following manners:

by an ultrasonic attenuation (U_{A}) hydrogel stability factor as described herein;

by a flow rate as described herein;

by a viscosity as described herein;

by a total osmolality as described herein;

by a phosphate ion concentration as described herein;

by a carbonate ion concentration as described herein;

by both a U_{A} hydrogel stability factor and flow rate as described herein;

by both a U_{A} hydrogel stability factor and viscosity as described herein;

by both a U_{A} hydrogel stability factor and total osmolality as described herein;

by both a U_{A} hydrogel stability factor and phosphate ion concentration as described herein;

by both a U_{A} hydrogel stability factor and carbonate ion concentration as described herein;

by both a flow rate and a viscosity as described herein;

by both a flow rate and total osmolality as described herein;

by both a flow rate and phosphate ion concentration as described herein;

by both a flow rate and carbonate ion concentration as described herein;

by both a viscosity and total osmolality as described herein;

by both a viscosity and phosphate ion concentration as described herein;

by both a viscosity and carbonate ion concentration as described herein;

by both a total osmolality and phosphate ion concentration as described herein;

by both a total osmolality and carbonate ion concentration as described herein;

by both a phosphate ion concentration and carbonate ion concentration as described herein;

by all of a U_{A} hydrogel stability factor, flow rate, and viscosity as described herein;

by all of a U_{A} hydrogel stability factor, flow rate, and total osmolality as described herein;

by all of a U_{A} hydrogel stability factor, flow rate, and phosphate ion concentration as described herein;

by all of a U_{A} hydrogel stability factor, flow rate, and carbonate ion concentration as described herein;

by all of a U_{A} hydrogel stability factor, viscosity, and total osmolality as described herein;

by all of a U_{A} hydrogel stability factor, viscosity, and phosphate ion concentration as described herein;

by all of a U_{A} hydrogel stability factor, viscosity, and carbonate ion concentration as described herein;

by all of a U_{A} hydrogel stability factor, total osmolality, and phosphate ion concentration as described herein;

by all of a U_{A} hydrogel stability factor, total osmolality, and carbonate ion concentration as described herein;

by all of a U_{A} hydrogel stability factor, phosphate ion concentration, and carbonate ion concentration as described herein;

by all of a flow rate, viscosity, and total osmolality as described herein;

by all of a flow rate, viscosity, and phosphate ion concentration as described herein;

by all of a flow rate, viscosity, and carbonate ion concentration as described herein;

by all of a flow rate, total osmolality, and phosphate ion concentration as described herein;

by all of a flow rate, total osmolality, and carbonate ion concentration as described herein;

by all of a flow rate, phosphate ion concentration, and carbonate ion concentration as described herein;

by all of a viscosity, total osmolality, and phosphate ion concentration as described herein;

by all of a viscosity, total osmolality, and carbonate ion concentration as described herein;

by all of viscosity, phosphate ion concentration, and carbonate ion concentration as described herein;

by all of a total osmolality, phosphate ion concentration, and carbonate ion concentration as described herein;

by all of a U_{A} hydrogel stability factor, flow rate, viscosity, and total osmolality as described herein;

by all of a U_{A} hydrogel stability factor, flow rate, viscosity, and phosphate ion concentration as described herein;

by all of a U_{A} hydrogel stability factor, flow rate, viscosity, and carbonate ion concentration as described herein;

by all of a U_{A} hydrogel stability factor, flow rate, and total osmolality, and phosphate ion concentration as described herein;

by all of a U_{A} hydrogel stability factor, flow rate, total osmolality, and carbonate ion concentration as described herein;

by all of a U_{A} hydrogel stability factor, flow rate, phosphate ion concentration, and carbonate ion concentration as described herein;

by all of a U_{A} hydrogel stability factor, viscosity, total osmolality, and phosphate ion concentration as described herein;

by all of a U_{A} hydrogel stability factor, viscosity, total osmolality, and carbonate ion concentration as described herein;

by all of a U_{A} hydrogel stability factor, viscosity, phosphate ion concentration, and carbonate ion concentration as described herein;

by all of a U_{A} hydrogel stability factor, total osmolality, phosphate ion concentration, and carbonate ion concentration as described herein;

by all of a flow rate, viscosity, total osmolality, and phosphate ion concentration as described herein;

by all of a flow rate, viscosity, total osmolality, and carbonate ion concentration as described herein;

by all of a flow rate, viscosity, phosphate ion concentration, and carbonate ion concentration as described herein;

by all of a flow rate, total osmolality, phosphate ion concentration, and carbonate ion concentration as described herein;

by all of a viscosity, total osmolality, phosphate ion concentration, and carbonate ion concentration as described herein;

by all of a U_{A} hydrogel stability factor, flow rate, viscosity, total osmolality, and phosphate ion concentration as described herein;

by all of U_{A} hydrogel stability factor, flow rate, viscosity, total osmolality, and carbonate ion concentration as described herein;

by all of a U_{A} hydrogel stability factor, flow rate, viscosity, phosphate ion concentration, and carbonate ion concentration as described herein;

by all of a U_{A} hydrogel stability factor, flow rate, total osmolality, phosphate ion concentration, and carbonate ion concentration as described herein;

by all of a U_{A} hydrogel stability factor, viscosity, total osmolality, phosphate ion concentration, and carbonate ion concentration as described herein;

by all of a flow rate, viscosity, total osmolality, phosphate ion concentration, and carbonate ion concentration as described herein; and by all of a U_{A} hydrogel stability factor, flow rate, viscosity, total osmolality, phosphate ion concentration, and carbonate ion concentration as described herein.

In one embodiment, the present disclosure provides a stable hydrogel composition comprising gelatin and a polymeric carbohydrate (e.g., dextran) in a combination such that: the gelatin comprises about 60% by weight or greater of the total weight of the combination of the gelatin and the polymeric carbohydrate present in the composition; the total concentration of the gelatin and the polymeric carbohydrate in the composition is about 50 mg/mL to about 400 mg/mL; the hydrogel composition is a flowable, injectable liquid at a temperature of 35° C. or greater and is a solid or semi-solid gel matrix at lower temperatures; and exhibits an ultrasonic attenuation (U_{A}) hydrogel stability factor of 0.4 for at least 500 minutes when tested at a temperature of 35° C. and a frequency of 2-8 MHz.

In one embodiment, the present disclosure provides a stable hydrogel composition comprising gelatin and a polymeric carbohydrate (e.g., dextran) in a combination such that: the gelatin comprises about 60% by weight or greater of the total weight of the combination of the gelatin and the polymeric carbohydrate present in the composition; the total concentration of the gelatin and the polymeric carbohydrate in the composition is about 50 mg/mL to about 400 mg/mL; the hydrogel composition is a flowable, injectable liquid at a temperature of 35° C. or greater and is a solid or semi-solid gel matrix at lower temperatures; and has a total osmolality of less than about 400 mOsm/kg, more particularly about 25 mOsm/kg to about 375 mOsm/kg. The same stable hydrogel composition may be defined by having a phosphate ion concentration of no greater than about 20 mM. The same stable hydrogel composition may be defined by having a carbonate ion concentration of no greater than about 20 mM.

In one embodiment, the present disclosure provides a stable hydrogel composition comprising gelatin and a polymeric carbohydrate (e.g., dextran) in a combination such that: the gelatin comprises about 60% by weight or greater of the total weight of the combination of the gelatin and the polymeric carbohydrate present in the composition; the total concentration of the gelatin and the polymeric carbohydrate in the composition is about 50 mg/mL to about 400 mg/mL; the hydrogel composition is a flowable, injectable liquid at a temperature of 35° C. or greater and is a solid or semi-solid gel matrix at lower temperatures; and has a phosphate ion concentration of no greater than about 20 mM and a carbonate ion concentration of no greater than about 20 mM.

In one embodiment, the present disclosure provides a flowable, injectable composition comprising gelatin and a polymeric carbohydrate (e.g., dextran), the composition being a hydrogel reconstituted from a lyophilized form and further including two or more additives selected from the group consisting of surfactants (e.g., polysorbates, such as Tween), hygroscopic excipients (e.g., polyols, such as glycerol, or salts, such as NaCl), and bulking agents (e.g., sugars, particularly disaccharides, such as dextrose); wherein the gelatin comprises about 60% by weight or greater of the total weight of the combination of the gelatin and the polymeric carbohydrate present in the reconstituted composition, and wherein the total concentration of the gelatin and the polymeric carbohydrate in the composition is about 50 mg/mL to about 400 mg/mL. The same stable hydrogel composition may be defined by a flow rate of about 10 μL/s or greater when forced from a syringe through a ⅝ inch long 25 gauge needle under a syringe plunger pressure of 5 N at a temperature of 35° C. to 39° C. The same stable hydrogel composition may be defined by a viscosity of about 1.5 Pa-s or less at a temperature of 35° C. to 39° C. The same stable hydrogel composition may be defined by having a total osmolality of less than about 400 mOsm/kg, more particularly about 25 mOsm/kg to about 375 mOsm/kg. The same stable hydrogel composition may be defined by having a phosphate ion concentration of no greater than about 20 mM. The same stable hydrogel composition may be defined by having a carbonate ion concentration of no greater than about 20 mM.

In one embodiment, the present disclosure provides a flowable, injectable composition comprising gelatin and a polymeric carbohydrate (e.g., dextran), the composition being a hydrogel reconstituted from a lyophilized form and further including a surfactant (e.g., polysorbates, such as Tween) and a hygroscopic excipient (e.g., polyols, such as glycerol, or salts, such as NaCl); wherein the gelatin comprises about 60% by weight or greater of the total weight of the combination of the gelatin and the polymeric carbohydrate present in the reconstituted composition, and wherein the total concentration of the gelatin and the polymeric carbohydrate in the composition is about 50 mg/mL to about 400 mg/mL.

In one embodiment, the present disclosure provides a flowable, injectable composition comprising gelatin and a polymeric carbohydrate (e.g., dextran), the composition being a hydrogel reconstituted from a lyophilized form and further including a surfactant (e.g., polysorbates, such as Tween) and a bulking agent (e.g., sugars, particularly disaccharides, such as dextrose); wherein the gelatin comprises about 60% by weight or greater of the total weight of the combination of the gelatin and the polymeric carbohydrate present in the reconstituted composition, and wherein the total concentration of the gelatin and the polymeric carbohydrate in the composition is about 50 mg/mL to about 400 mg/mL.

In one embodiment, the present disclosure provides a flowable, injectable composition comprising gelatin and a polymeric carbohydrate (e.g., dextran), the composition being a hydrogel reconstituted from a lyophilized form and further including a hygroscopic excipient (e.g., polyols, such as glycerol, or salts, such as NaCl) and a bulking agent (e.g., sugars, particularly disaccharides, such as dextrose); wherein the gelatin comprises about 60% by weight or greater of the total weight of the combination of the gelatin and the polymeric carbohydrate present in the reconstituted composition, and wherein the total concentration of the gelatin and the polymeric carbohydrate in the composition is about 50 mg/mL to about 400 mg/mL.

In one embodiment, the present disclosure provides a flowable, injectable composition comprising gelatin and a polymeric carbohydrate (e.g., dextran), the composition being a hydrogel reconstituted from a lyophilized form and further including a surfactant (e.g., polysorbates, such as Tween), a hygroscopic excipient (e.g., polyols, such as glycerol, or salts, such as NaCl), and a bulking agent (e.g., sugars, particularly disaccharides, such as dextrose); wherein the gelatin comprises about 60% by weight or greater of the total weight of the combination of the gelatin and the polymeric carbohydrate present in the reconstituted composition, and wherein the total concentration of the gelatin and the polymeric carbohydrate in the composition is about 50 mg/mL to about 400 mg/mL.

In one embodiment, the present disclosure provides a method for preventing or reducing cutaneous scarring, or an effect thereof, arising from a cutaneous wound, the method comprising applying a flowable hydrogel composition to the cutaneous wound, wherein the flowable hydrogel composition comprises gelatin and a polymeric carbohydrate (e.g., dextran) in a combination such that: the gelatin comprises about 60% by weight or greater of the total weight of the combination of the gelatin and the polymeric carbohydrate present in the composition; the total concentration of the gelatin and the polymeric carbohydrate in the composition is about 50 mg/mL to about 400 mg/mL; the hydrogel composition is a flowable, injectable liquid at a temperature of 35° C. or greater and is a solid or semi-solid gel matrix at lower temperatures; and exhibits an ultrasonic attenuation (U_{A}) hydrogel stability factor of 0.4 for at least 500 minutes when tested at a temperature of 35° C. and a frequency of 2-8 MHz. The step of applying may comprise injecting the hydrogel composition into dermal or subdermal tissue. The same method may particularly be used for excision of a pre-existing scar, particularly a keloid, hypertrophic scar, or burn-related scar.

In one embodiment, the present disclosure provides a method for preventing or reducing cutaneous scarring, or an effect thereof, arising from a cutaneous wound, the method comprising applying a flowable hydrogel composition to the cutaneous wound, wherein the flowable hydrogel composition comprises gelatin and a polymeric carbohydrate (e.g., dextran) in a combination such that: the gelatin comprises about 60% by weight or greater of the total weight of the combination of the gelatin and the polymeric carbohydrate present in the composition; the total concentration of the gelatin and the polymeric carbohydrate in the composition is about 50 mg/mL to about 400 mg/mL; the hydrogel composition is a flowable, injectable liquid at a temperature of 35° C. or greater and is a solid or semi-solid gel matrix at lower temperatures; and has a total osmolality of less than about 400 mOsm/kg, more particularly about 25 mOsm/kg to about 375 mOsm/kg. The same stable hydrogel composition may be defined by having a phosphate ion concentration of no greater than about 20 mM. The same stable hydrogel composition may be defined by having a carbonate ion concentration of no greater than about 20 mM. The step of applying may comprise injecting the hydrogel composition into dermal or subdermal tissue. The same method may particularly be used for excision of a pre-existing scar, particularly a keloid, hypertrophic scar, or burn-related scar.

In one embodiment, the present disclosure provides a method for preventing or reducing cutaneous scarring, or an effect thereof, arising from a cutaneous wound, the method comprising applying a flowable hydrogel composition to the cutaneous wound, wherein the flowable hydrogel composition comprises gelatin and a polymeric carbohydrate (e.g., dextran) in a combination such that: the gelatin comprises about 60% by weight or greater of the total weight of the combination of the gelatin and the polymeric carbohydrate present in the composition; the total concentration of the gelatin and the polymeric carbohydrate in the composition is about 50 mg/mL to about 400 mg/mL; the hydrogel composition is a flowable, injectable liquid at a temperature of 35° C. or greater and is a solid or semi-solid gel matrix at lower temperatures; and has a phosphate ion concentration of no greater than about 20 mM and a carbonate ion concentration of no greater than about 20 mM. The step of applying may comprise injecting the hydrogel composition into dermal or subdermal tissue. The same method may be used for excision of a pre-existing scar, particularly a keloid, hypertrophic scar, or burn-related scar.

In one embodiment, the present disclosure provides a method for preventing or reducing cutaneous scarring, or an effect thereof, arising from a cutaneous wound, the method comprising applying a flowable hydrogel composition to the cutaneous wound, wherein the flowable hydrogel composition comprises gelatin and a polymeric carbohydrate (e.g., dextran), the composition being a hydrogel reconstituted from a lyophilized form and further including two or more additives selected from the group consisting of surfactants (e.g., polysorbates, such as Tween), hygroscopic excipients (e.g., polyols, such as glycerol, or salts, such as NaCl), and bulking agents (e.g., sugars, particularly disaccharides, such as dextrose); wherein the gelatin comprises about 60% by weight or greater of the total weight of the combination of the gelatin and the polymeric carbohydrate present in the reconstituted composition, and wherein the total concentration of the gelatin and the polymeric carbohydrate in the composition is about 50 mg/mL to about 400 mg/mL. The step of applying may comprise injecting the hydro gel composition into dermal or subdermal tissue. The same method may particularly be used for excision of a pre-existing scar, particularly a keloid, hypertrophic scar, or burn-related scar.

In one embodiment, the present disclosure provides a method for preventing or reducing cutaneous scarring, or an effect thereof, arising from a cutaneous wound, the method comprising applying a flowable hydrogel composition to the cutaneous wound, wherein the flowable hydrogel composition comprises gelatin and a polymeric carbohydrate (e.g., dextran), the composition being a hydrogel reconstituted from a lyophilized form and further including a surfactant (e.g., polysorbates, such as Tween) and a hygroscopic excipient (e.g., polyols, such as glycerol, or salts, such as NaCl); wherein the gelatin comprises about 60% by weight or greater of the total weight of the combination of the gelatin and the polymeric carbohydrate present in the reconstituted composition, and wherein the total concentration of the gelatin and the polymeric carbohydrate in the composition is about 50 mg/mL to about 400 mg/mL. The step of applying may comprise injecting the hydrogel composition into dermal or subdermal tissue. The same method may particularly be used for excision of a pre-existing scar, particularly a keloid, hypertrophic scar, or burn-related scar.

In one embodiment, the present disclosure provides a method for preventing or reducing cutaneous scarring, or an effect thereof, arising from a cutaneous wound, the method comprising applying a flowable hydrogel composition to the cutaneous wound, wherein the flowable hydrogel composition comprises gelatin and a polymeric carbohydrate (e.g., dextran), the composition being a hydrogel reconstituted from a lyophilized form and further including a surfactant (e.g., polysorbates, such as Tween) and a bulking agent (e.g., sugars, particularly disaccharides, such as dextrose); wherein the gelatin comprises about 60% by weight or greater of the total weight of the combination of the gelatin and the polymeric carbohydrate present in the reconstituted composition, and wherein the total concentration of the gelatin and the polymeric carbohydrate in the composition is about 50 mg/mL to about 400 mg/mL. The step of applying may comprise injecting the hydrogel composition into dermal or subdermal tissue. The same method may particularly be used for excision of a pre-existing scar, particularly a keloid, hypertrophic scar, or burn-related scar.

In one embodiment, the present disclosure provides a method for preventing or reducing cutaneous scarring, or an effect thereof, arising from a cutaneous wound, the method comprising applying a flowable hydrogel composition to the cutaneous wound, wherein the flowable hydrogel composition comprises gelatin and a polymeric carbohydrate (e.g., dextran), the composition being a hydrogel reconstituted from a lyophilized form and further including a hygroscopic excipient (e.g., polyols, such as glycerol, or salts, such as NaCl) and a bulking agent (e.g., sugars, particularly disaccharides, such as dextrose); wherein the gelatin comprises about 60% by weight or greater of the total weight of the combination of the gelatin and the polymeric carbohydrate present in the reconstituted composition, and wherein the total concentration of the gelatin and the polymeric carbohydrate in the composition is about 50 mg/mL to about 400 mg/mL. The step of applying may comprise injecting the hydrogel composition into dermal or subdermal tissue. The same method may particularly be used for excision of a pre-existing scar, particularly a keloid, hypertrophic scar, or burn-related scar.

In one embodiment, the present disclosure provides a method for preventing or reducing cutaneous scarring, or an effect thereof, arising from a cutaneous wound, the method comprising applying a flowable hydrogel composition to the cutaneous wound, wherein the flowable hydrogel composition comprises gelatin and a polymeric carbohydrate (e.g., dextran), the composition being a hydrogel reconstituted from a lyophilized form and further including a surfactant (e.g., polysorbates, such as Tween), a hygroscopic excipient (e.g., polyols, such as glycerol, or salts, such as NaCl), and a bulking agent (e.g., sugars, particularly disaccharides, such as dextrose); wherein the gelatin comprises about 60% by weight or greater of the total weight of the combination of the gelatin and the polymeric carbohydrate present in the reconstituted composition, and wherein the total concentration of the gelatin and the polymeric carbohydrate in the composition is about 50 mg/mL to about 400 mg/mL. The step of applying may comprise injecting the hydrogel composition into dermal or subdermal tissue. The same method may particularly be used for excision of a pre-existing scar, particularly a keloid, hypertrophic scar, or burn-related scar.

In one embodiment, the present disclosure provides a method revision of a keloid, the method comprising excising at least a portion of the keloid and applying a flowable hydrogel composition to at least a portion of the excision site, wherein the flowable hydrogel composition comprises gelatin and a polymeric carbohydrate (e.g., dextran) in a combination such that: the gelatin comprises about 60% by weight or greater of the total weight of the combination of the gelatin and the polymeric carbohydrate present in the composition; the total concentration of the gelatin and the polymeric carbohydrate in the composition is about 50 mg/mL to about 400 mg/mL; and the hydrogel composition is a flowable, injectable liquid at a temperature of 35° C. or greater and is a solid or semi-solid gel matrix at lower temperatures.

In one embodiment, the present disclosure provides a method revision of a keloid, the method comprising excising at least a portion of the keloid and applying a flowable hydrogel composition to at least a portion of the excision site, wherein the flowable hydrogel composition comprises gelatin and a polymeric carbohydrate (e.g., dextran) in a combination such that: the gelatin comprises about 60% by weight or greater of the total weight of the combination of the gelatin and the polymeric carbohydrate present in the composition; the total concentration of the gelatin and the polymeric carbohydrate in the composition is about 50 mg/mL to about 400 mg/mL; the hydrogel composition is a flowable, injectable liquid at a temperature of 35° C. or greater and is a solid or semi-solid gel matrix at lower temperatures; and the composition comprises two or more additives selected from the group consisting of surfactants (e.g., polysorbates, such as Tween), hygroscopic excipients (e.g., polyols, such as glycerol, or salts, such as NaCl), and bulking agents (e.g., sugars, particularly disaccharides, such as dextrose).

In one embodiment, the present disclosure provides a method revision of a keloid, the method comprising excising at least a portion of the keloid and applying a flowable hydrogel composition to at least a portion of the excision site, wherein the flowable hydrogel composition comprises gelatin and a polymeric carbohydrate (e.g., dextran) in a combination such that: the gelatin comprises about 60% by weight or greater of the total weight of the combination of the gelatin and the polymeric carbohydrate present in the composition; the total concentration of the gelatin and the polymeric carbohydrate in the composition is about 50 mg/mL to about 400 mg/mL; the hydrogel composition is a flowable, injectable liquid at a temperature of 35° C. or greater and is a solid or semi-solid gel matrix at lower temperatures; and the composition exhibits an ultrasonic attenuation (U_{A}) hydrogel stability factor of 0.4 for at least 500 minutes when tested at a temperature of 35° C. and a frequency of 2-8 MHz.

In one embodiment, the present disclosure provides a method revision of a keloid, the method comprising excising at least a portion of the keloid and applying a flowable hydrogel composition to at least a portion of the excision site, wherein the flowable hydrogel composition comprises gelatin and a polymeric carbohydrate (e.g., dextran) in a combination such that: the gelatin comprises about 60% by weight or greater of the total weight of the combination of the gelatin and the polymeric carbohydrate present in the composition; the total concentration of the gelatin and the polymeric carbohydrate in the composition is about 50 mg/mL to about 400 mg/mL; the hydrogel composition is a flowable, injectable liquid at a temperature of 35° C. or greater and is a solid or semi-solid gel matrix at lower temperatures; and the composition has a total osmolality of less than about 400 mOsm/kg, more particularly about 25 mOsm/kg to about 375 mOsm/kg.

In one embodiment, the present disclosure provides a method revision of a keloid, the method comprising excising at least a portion of the keloid and applying a flowable hydrogel composition to at least a portion of the excision site, wherein the flowable hydrogel composition comprises gelatin and a polymeric carbohydrate (e.g., dextran) in a combination such that: the gelatin comprises about 60% by weight or greater of the total weight of the combination of the gelatin and the polymeric carbohydrate present in the composition; the total concentration of the gelatin and the polymeric carbohydrate in the composition is about 50 mg/mL to about 400 mg/mL; the hydrogel composition is a flowable, injectable liquid at a temperature of 35° C. or greater and is a solid or semi-solid gel matrix at lower temperatures; and the composition a phosphate ion concentration of no greater than about 20 mM and a carbonate ion concentration of no greater than about 20 mM.

In one embodiment, the present disclosure provides a method of preparing a stable hydrogel composition, the method comprising: mixing gelatin and a polymeric carbohydrate in an aqueous medium at a first temperature to form a liquid hydrogel composition wherein the gelatin comprises about 60% by weight or greater of the total weight of the combination of the gelatin and the polymeric carbohydrate present in the composition; cooling the liquid hydrogel composition with constant mixing to a holding temperature that is less than the first temperature and greater than the gelation temperature at which the liquid hydrogel composition transitions to a solid or semi-solid hydrogel composition; and further cooling the liquid hydrogel composition to the gelation temperature to transition the liquid hydrogel composition to a stable, solid or semi-solid hydrogel composition exhibiting an ultrasonic attenuation (U_{A}) hydrogel stability factor of 0.4 for at least 500 minutes when tested at a temperature of 35° C. and a frequency of 2-8 MHz, or a U_{A} hydrogel stability factor of 0.5 for at least 600 minutes when tested at a temperature of 35° C. and a frequency of 2-8 MHz; or a U_{A} hydrogel stability factor of 0.3 for at least 500 minutes when tested at a temperature of 35° C. and a frequency of 2.7 MHz; or a U_{A} hydrogel stability factor of 0.2 for at least 500 minutes when tested at a temperature of 35° C. and a frequency of 5.1 MHz; or a U_{A} hydrogel stability factor of 0.35 for at least 500 minutes when tested at a temperature of 35° C. and a frequency of 7.8 MHz.

In one embodiment, the present disclosure provides a method of preparing a stable hydrogel composition, the method comprising: mixing gelatin and a polymeric carbohydrate in an aqueous medium at a first temperature to form a liquid hydrogel composition wherein the gelatin comprises about 60% by weight or greater of the total weight of the combination of the gelatin and the polymeric carbohydrate present in the composition; cooling the liquid hydrogel composition with constant mixing to a holding temperature that is at least about 5° C. less than the first temperature and is within about 7° C. of the gelation temperature at which the liquid hydrogel composition transitions to a solid or semi-solid hydrogel composition; and further cooling the liquid hydrogel composition to the gelation temperature to transition the liquid hydrogel composition to a stable, solid or semi-solid hydrogel composition.

In one embodiment, the present disclosure provides a method of preparing a stable hydrogel composition, the method comprising: mixing gelatin and a polymeric carbohydrate in an aqueous medium at a first temperature to form a liquid hydrogel composition wherein the gelatin comprises about 60% by weight or greater of the total weight of the combination of the gelatin and the polymeric carbohydrate present in the composition and wherein the total concentration of the gelatin and the polymeric carbohydrate in the stable hydrogel composition is about 50 mg/mL to about 400 mg/mL; cooling the liquid hydrogel composition with constant mixing to a holding temperature that is at least about 5° C. less than the first temperature and is within about 7° C. of the gelation temperature at which the liquid hydrogel composition transitions to a solid or semi-solid hydrogel composition; and further cooling the liquid hydrogel composition to the gelation temperature to transition the liquid hydrogel composition to a stable, solid or semi-solid hydrogel composition, wherein further cooling to the gelation temperature is carried out in a time of less than about 2 hours.

In one embodiment, the present disclosure provides a method of preparing a stable hydrogel composition, the method comprising: mixing gelatin and a polymeric carbohydrate in an aqueous medium having an osmolality of less than about 400 mOsm/kg (e.g., about 25 mOsm/kg to about 375 mOsm/kg) at a first temperature to form a liquid hydrogel composition wherein the gelatin comprises about 60% by weight or greater of the total weight of the combination of the gelatin and the polymeric carbohydrate present in the composition and wherein the total concentration of the gelatin and the polymeric carbohydrate in the stable hydrogel composition is about 50 mg/mL to about 400 mg/mL; cooling the liquid hydrogel composition with constant mixing to a holding temperature that is less than the first temperature and greater than the gelation temperature at which the liquid hydro gel composition transitions to a solid or semi-solid hydrogel composition; and further cooling the liquid hydrogel composition to the gelation temperature to transition the liquid hydrogel composition to a stable, solid or semi-solid hydrogel composition.

In one embodiment, the present disclosure provides a method of preparing a stable hydrogel composition, the method comprising: mixing gelatin and a polymeric carbohydrate in an aqueous medium having a phosphate ion concentration of no greater than about 20 mM and a carbonate ion concentration of no greater than about 20 mM at a first temperature to form a liquid hydrogel composition wherein the gelatin comprises about 60% by weight or greater of the total weight of the combination of the gelatin and the polymeric carbohydrate present in the composition and wherein the total concentration of the gelatin and the polymeric carbohydrate in the stable hydrogel composition is about 50 mg/mL to about 400 mg/mL; cooling the liquid hydrogel composition with constant mixing to a holding temperature that is less than the first temperature and greater than the gelation temperature at which the liquid hydrogel composition transitions to a solid or semi-solid hydrogel composition; and further cooling the liquid hydrogel composition to the gelation temperature to transition the liquid hydrogel composition to a stable, solid or semi-solid hydrogel composition.

In one embodiment, the present disclosure provides a method of preparing a hydrogel composition, the method comprising: providing a lyophilized composition comprising gelatin and a polymeric carbohydrate, the gelatin comprising about 60% by weight or greater of the total weight of the combination of the gelatin and the polymeric carbohydrate present in the lyophilized composition; and reconstituting the lyophilized composition with an aqueous reconstitution fluid to form the hydrogel composition such that the reconstituted hydrogel composition includes two or more additives selected from the group consisting of surfactants, hygroscopic excipients, and bulking agents, and such that the total concentration of the gelatin and the polymeric carbohydrate in the reconstituted hydrogel composition is about 50 mg/mL to about 400 mg/mL; wherein the aqueous reconstitution fluid in contact with the lyophilized composition comprises about 0.01% to about 4% by weight of the surfactant.

In one embodiment, the present disclosure provides a method of preparing a hydrogel composition, the method comprising: providing a lyophilized composition comprising gelatin and a polymeric carbohydrate, the gelatin comprising about 60% by weight or greater of the total weight of the combination of the gelatin and the polymeric carbohydrate present in the lyophilized composition; and reconstituting the lyophilized composition with an aqueous reconstitution fluid to form the hydrogel composition such that the reconstituted hydrogel composition includes two or more additives selected from the group consisting of surfactants, hygroscopic excipients, and bulking agents, and such that the total concentration of the gelatin and the polymeric carbohydrate in the reconstituted hydrogel composition is about 50 mg/mL to about 400 mg/mL; wherein the aqueous reconstitution fluid in contact with the lyophilized composition comprises about 0.01% to about 4% by weight of the hygroscopic excipient.

In one embodiment, the present disclosure provides a method of preparing a hydrogel composition, the method comprising: providing a lyophilized composition comprising gelatin and a polymeric carbohydrate, the gelatin comprising about 60% by weight or greater of the total weight of the combination of the gelatin and the polymeric carbohydrate present in the lyophilized composition; and reconstituting the lyophilized composition with an aqueous reconstitution fluid to form the hydrogel composition such that the reconstituted hydrogel composition includes two or more additives selected from the group consisting of surfactants, hygroscopic excipients, and bulking agents, and such that the total concentration of the gelatin and the polymeric carbohydrate in the reconstituted hydrogel composition is about 50 mg/mL to about 400 mg/mL; wherein the aqueous reconstitution fluid in contact with the lyophilized composition comprises about 0.01% to about 4% by weight of the bulking agent.

In one embodiment, the present disclosure provides a method of preparing a hydrogel composition, the method comprising: providing a lyophilized composition comprising gelatin and a polymeric carbohydrate, the gelatin comprising about 60% by weight or greater of the total weight of the combination of the gelatin and the polymeric carbohydrate present in the lyophilized composition; and reconstituting the lyophilized composition with an aqueous reconstitution fluid to form the hydrogel composition such that the reconstituted hydrogel composition includes any of: i) a surfactant and a hygroscopic excipient; ii) a surfactant and a bulking agent; iii) a hygroscopic excipient and a bulking agent; and iv) a surfactant, a hygroscopic excipient, and a bulking agent; and such that the total concentration of the gelatin and the polymeric carbohydrate in the reconstituted hydrogel composition is about 50 mg/mL to about 400 mg/mL.

In one embodiment, the present disclosure provides a method of preparing a hydrogel composition, the method comprising: providing a lyophilized composition comprising gelatin and a polymeric carbohydrate, the gelatin comprising about 60% by weight or greater of the total weight of the combination of the gelatin and the polymeric carbohydrate present in the lyophilized composition; and reconstituting the lyophilized composition with an aqueous reconstitution fluid to form the hydrogel composition such that the reconstituted hydrogel composition includes any of: i) a polysorbate and a polyol; ii) a polysorbate and a salt; iii) a polysorbate and a sugar; iv) a polyol and a sugar; v) a salt and a sugar; vi) a polysorbate, a polyol, and a sugar; and vii) a polysorbate, a salt, and a sugar; and such that the total concentration of the gelatin and the polymeric carbohydrate in the reconstituted hydrogel composition is about 50 mg/mL to about 400 mg/mL

In one embodiment, the present disclosure provides a method of preparing a hydrogel composition, the method comprising: providing a lyophilized composition comprising gelatin and a polymeric carbohydrate, the gelatin comprising about 60% by weight or greater of the total weight of the combination of the gelatin and the polymeric carbohydrate present in the lyophilized composition; and reconstituting the lyophilized composition with an aqueous reconstitution fluid to form the hydro gel composition such that the reconstituted hydrogel composition includes any of: i) Tween and glycerol; ii) Tween and NaCl; iii) Tween and a disaccharide; iv) glycerol and a disaccharide; v) NaCl and a disaccharide; vi) Tween, glycerol, and a disaccharide; and vii) Tween, NaCl, and a disaccharide; and such that the total concentration of the gelatin and the polymeric carbohydrate in the reconstituted hydrogel composition is about 50 mg/mL to about 400 mg/mL.

### Examples

The present invention is more fully illustrated below by the following examples, which are set forth to illustrate embodiments of the present invention and are not to be construed as limiting thereof.

### Example 1

### Preparation of Gelatin/Dextran Composition

A composition according one embodiment of the present disclosure was prepared by sequentially adding the liquid and powdered raw materials (Table 1) at specific time points at elevated temperatures, while mixed to maintain a liquid, homogeneous state. The solution was then sterile filtered, aseptically dispensed into vials, sealed, and stored at refrigerated temperatures.

TABLE 1

| Component | Concentration | |
|---|---|---|
| Gelatin - Type-A porcine gelatin | 120 | mg/ml |
| 100,000 Da Avg. MW | | |
| Dextran 500,000 Da Avg. MW | 50 | mg/ml |
| Medium 199 Custom Formulation | 0.83 | ml/ml |
| C5866 | | |
| L-Glutamic Acid, Monosodium Salt, | 3.74 | mg/ml |
| N.F. | | |
| L-Arginine Monohydrochloride, USP | 3.16 | mg/ml |
| Edetate Disodium, USP | 1.46 | mg/ml |
| L-Lysine Acetate, USP | 1.03 | mg/ml |
| L-Cysteine HCl Injection, USP | 0.13 | mg/ml |
| Zinc Sulfate, USP | 0.005 | mg/ml |
| L-Alanyl-L-Glutamine | 0.002 | mg/ml |
| 50% Dextrose Injection, USP | 0.50 | mg/ml |

| | |
|---|---|
| Hydrochloric Acid, USP | As needed to adjust pH |
| Sodium Hydroxide, NF | As needed to adjust pH |
| Sterile Water for Injection, USP | As needed to adjust osmolality |

The dextrose and Medium 199 (with a phosphate ion concentration of 1.17 mM) were measured and transferred to a pre-heated, water-jacketed (50° C.) glass vessel and mixed using a stir bar. The mixture was allowed to equilibrate to 50° C. after which L-Cysteine, L-Alanyl-L-Glutamine, L-Glutamic Acid, L-Lysine, and EDTA Disodium were dispensed into the vessel with Medium 199. After an equilibration time, dextran powder was added, and the components were mixed. Once the dextran appeared to be in solution, gelatin was added to the vessel and allowed to dissolve. Once the gelatin appeared to be in solution, pH was adjusted to 7.45+/−0.05. After equilibration from the pH adjustment, the remaining L-Glutamic Acid, Arginine, and L-Cysteine were stirred into the solution along with the Zinc Sulfate. The total mixing time was approximately 2 hours.

After equilibration at 50° C., the solution was filter sterilized by pumping the solution from the mixing vessel through a heated, 0.2 μm positively charged, pharmaceutical grade nylon filter into a 40° C. stirring vessel for queuing prior to aseptic filling. Filtration was performed in approximately 30 minutes. After the solution was filtered, it was aseptically dispensed into 12 ml aliquots into vials while it continued to be mixed while maintained at 40° C. The vials were subsequently stoppered, sealed, crimped, visually inspected, labeled, and then stored under refrigeration (2-8° C.). The production yielded approximately 150 vials. The dispensing of the composition was performed in approximately one hour.

### Example 2

### Preparation of Gelatin/Dextran Composition with Phosphate Buffer

A composition was prepared by sequentially adding the liquid and powdered raw materials (Table 2) at elevated temperatures while being mixed to maintain a liquid, homogeneous state.

TABLE 2

| Component | Concentration | |
|---|---|---|
| Gelatin - Type-A porcine gelatin | 120 | mg/ml |
| 100,000 Da Avg. MW | | |
| Dextran 500,000 Da Avg. MW | 50 | mg/ml |
| Phosphate Buffered Saline | 0.83 | ml/ml |
| L-Glutamic Acid, Monosodium Salt, | 3.74 | mg/ml |
| N.F. | | |
| L-Arginine Monohydrochloride, USP | 3.16 | mg/ml |
| Edetate Disodium, USP | 1.46 | mg/ml |
| L-Lysine Acetate, USP | 1.03 | mg/ml |
| L-Cysteine HCl Injection, USP | 0.13 | mg/ml |
| Zinc Sulfate, USP | 0.005 | mg/ml |

| | |
|---|---|
| Hydrochloric Acid, USP | As needed to adjust pH |
| Sodium Hydroxide, NF | As needed to adjust pH |
| Sterile Water for Injection, USP | As needed to adjust osmolality |

Unlike the composition of Example 1, the formulation of the present composition was prepared using phosphate buffered saline (PBS) with a phosphate ion concentration of 65 mM. Additionally, no L-Alynyl-L-Glutamine or 50% Dextrose were added in the present example. Specifically, the composition of the present example was prepared by first placing 2,282 ml of PBS into a pre-heated, water-jacketed (50° C.) glass vessel and mixed using a stir bar. The mixture was allowed to equilibrate to 50° C. after which the 375 μl of L-Cysteine, 6.9 ml of L-Glutamic Acid, 13.8 ml of L-Lysine, and 24.2 ml of EDTA Disodium were dispensed into the vessel with PBS. After an equilibration time, 137.5 grams of dextran powder was added and the components were mixed. Once the dextran appeared to be in solution, 330 grams of gelatin was added to the vessel and allowed to dissolve. Once the gelatin appeared to be in solution, 10% sodium hydroxide was added to adjust the pH to 7.45+/−0.05. After equilibration from the pH adjustment, 20.6 ml of L-Glutamic Acid, 20.6 ml of Arginine, 13.8 ml of Zinc Sulfate, and 6.5 ml of L-Cysteine were stirred into the solution. The vials were subsequently stoppered, sealed, crimped, visually inspected, labeled, and then stored under refrigeration (2-8° C.). The total mixing time was approximately 2 hours.

### Example 3

### Thermal Properties of Composition

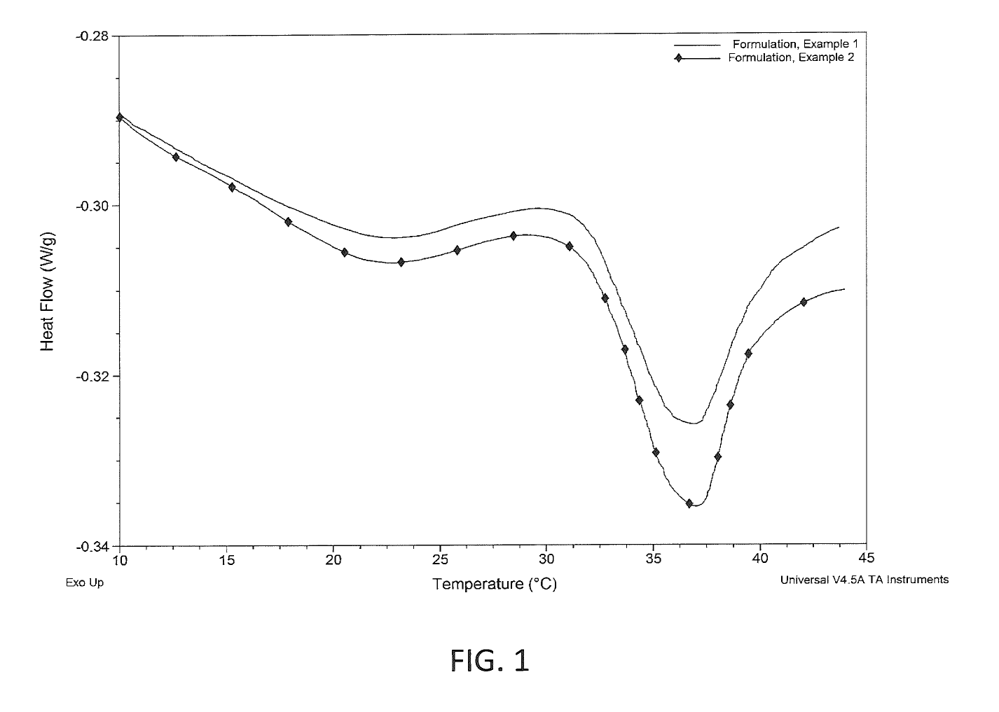

The thermal properties of the compositions from Example 1 and Example 2 were examined by differential scanning calorimetry (DSC). Samples of the compositions from Examples 1 and 2 were incubated for 60 minutes at 5° C. and then subjected to DSC testing at a scan rate of 5° C. per minute, starting from 5° C. to an ending temperature of 45° C. The thermal properties were evaluated from the representative thermagrams and enthalpy of thermal transitions. A melt transition of the solid composition to a liquid was observed, with a transition peak ranging from 35 to 37° C. A representative thermogram for the composition is shown in FIG. 1.

### Example 4

### Flowability/Injectability of Composition

A composition of 60% gelatin and 40% dextran was solubilized to a final solids concentration of 0.2 g/ml in Tris buffered saline (TBS) heated to 50° C. The resulting composition was divided into 4 ml samples and placed in 20 ml vials, and the vials with the samples were stored at 4° C. To evaluate flowability, a vial of the composition was removed from storage and placed at room temperature. The vial was then placed in a heater (Lab-line Multiblock Heater) at 39° C. and monitored until the composition transitioned from a solid to a flowable liquid. The liquid composition was placed into a 1 ml syringe and attached to a fluid dispensing system (1500 XL, Nordson/EFD) set to a plunger force of 5 N, the recommended force for a 1 ml syringe by the international guidance for sterile hypodermic syringes for single use (see, ISO 7886-1). A 25 gauge needle (0.26 mm nominal inner diameter) of approximately 16 mm (⅝ inch) length was attached and the time for passage of 1 ml of composition through the needle was recorded. Injection through the needle was carried out at a temperature of about 39° C. The composition met the conditions for administration by injection through a fine gauge needle according to ISO 7886-1, which specifies requirements (including performance) for sterile, single-use hypodermic syringes made of plastic materials and intended for the aspiration of fluids or for the injection of fluids immediately after filling.

To examine the solids content of the composition to evaluate suitability for injection through a small gauge needle, the total polymer content of dextran and gelatin in the composition was varied as presented in Table 3. The polymers were solubilized in 10 ml of HEPES buffered saline (0.01M HEPES, 0.138 M NaCl, and 0.0027 M KCl) with slow mixing in 15 ml conical tubes at 50° C.

TABLE 3

| Total Polymer | Gelatin | Dextran |
|---|---|---|
| (mg/mL) | (mg/mL) | (mg/mL) |
| 190 | 120 | 70 |
| 304 | 192 | 112 |
| 380 | 240 | 140 |
| 427.5 | 270 | 157.5 |
| 475 | 300 | 175 |

After preparation the samples were cooled. Using the testing methods described above, the samples were placed in a 39° C. heating block and 1 ml was injected through 25 gauge and 23 gauge needles that were one inch long (22.5 mm). Each sample was tested a minimum of three times through each needle size for injection rate. If a minimum of 0.5 ml was not able to be injected through the needle, the test was unsuccessful, and the sample continued to be heated until successfully injected. The results of the injection testing are presented in Table 4.

TABLE 4

| Total Polymer | Approximate Melt | 23 G Avg. Rate | 25 G Avg. Rate |
|---|---|---|---|
| (mg/mL) | Time (min) | (μL/sec) | (μL/sec) |
| 190 | 8 | 63 | 29 |
| 304 | 10 | 15.4 | 5.7 |
| 380 | 8 | 7.6 | 2.3 |
| 427.5 | 14 | 3.5 | unable to inject |
| 475 | 18 | 1.6 | unable to inject |

The formulations tested for injectability were characterized for viscosity to determine the maximum viscosity to allow injection. Sample formulations using the same ratio of gelatin to dextran tested for injectability were prepared at various total polymer content in HEPES buffered saline as previously described. The samples were preconditioned at 50° C. and loaded into a Rheometrics Scientific RFSII Rheometer with Couette Geometry for testing. Multiple measurements were made with freshly loaded samples at 39° C. until viscosity stabilized to equilibrium measurement values. A shear rate range up to 1000 s⁻¹ was tested; however, the shear rate range for each sample was adjusted to the torque range of the rheometer. Shear rate range was reduced for high concentration samples.

The samples from 190 to 380 mg/mL total polymer demonstrated Newtonian viscosity response. The higher concentration samples demonstrated slight non-linear viscosity response but were suitable for characterization by standard linear regression analysis. The viscosity results are summarized in Table 5. Formulation viscosity greater than 0.4565 Pa-s was not suitable for injection through a 25 gauge needle. Viscosity of up to 1.1618 Pa-s was injectable through a 23 gauge needle.

TABLE 5

| Total Polymer | Viscosity | Viscosity |
|---|---|---|
| (mg/mL) | Avg (Pa-s) | Std Dev (Pa-s) |
| 190 | 0.0598 | 0.00045 |
| 285 | 0.1983 | 0.03579 |
| 380 | 0.4565 | 0.02748 |
| 427.5 | 0.7514 | 0.03334 |
| 475 | 1.1618 | 0.02666 |

### Example 5

### Fibronectin Binding Activity of Composition

The composition of Example 1 was evaluated in relation to fibronectin binding activity of the gelatin. The assay followed a direct ELISA binding format where test material was immobilized on well surfaces of a microtiter plate to bind fluoresceinated human plasma fibronectin to enable direct measurement of fluorescence of the captured tagged fibronectin. In this assay, the composition of Example 1 was completely melted (by incubation at 39° C. for 30 minutes), and diluted with pH 9.6 buffer to yield final gelatin concentration in the range of 1 to 10 μg/mL to coat wells of a high-binding opaque 96-well plate, optimal for fluorescence measurement. The gelatin used in the composition was immobilized irreversibly onto the well surfaces by incubation of the plate at 39° C. for one hour. Excess unbound materials and buffers were removed, and the plate was washed three times with PBS (Phosphate Buffered Saline) containing 1M NaCl (Wash Buffer). Nonspecific binding sites were blocked by 3% milk in PBS (Blocker) for 30 minutes at room temperature. Excess buffer was removed and the plate was washed 3 times with Wash Buffer as performed previously. Human plasma fibronectin, conjugated with 1 to 5 fluorescein molecules per fibronectin molecule, diluted with Blocker to a final concentration of 45 μg/mL, was then added to the plate wells. The plate was covered and rotated gently at room temperature for 2 hrs to allow maximal binding in the dark. Excess fluoresceinated fibronectin was removed, and the plate was washed 3 times with Wash Buffer as performed previously. PBS (100 μL) was added to each well, and fluorescence was recorded by a plate reader, using excitation wavelength at 485 nm and emission wavelength at 530 nm. Plates were read several times, and the data averaged.

Both samples and standards were measured in duplicates. The gelatin raw material used in the composition was tested at seven concentrations (see Table 6) to generate a standard curve. The composition samples were tested at four diluted concentrations. Concentrations of gelatins in each sample were determined from the range of standards by linear regression.

TABLE 6

| | Average Relative Fluoresence |
|---|---|
| Gelatin (μg) | Units, background corrected |
| 1 | 7.24 |
| 0.8 | 6.75 |
| 0.5 | 5.57 |
| 0.4 | 4.59 |
| 0.25 | 2.86 |
| 0.2 | 2.05 |
| 0.125 | 0.97 |
| 0 | 0 |

In order to compare the fibronectin binding content of the composition, the protein content was also measured and used to normalize the amount of fibronectin binding sites of each sample. Protein concentration was measured by the colorimetric Bicinchoninic Acid (BCA) assay (Pierce Biochemical Micro BCA protein assay kit, #23235) according to the manufacturer's instructions. Briefly, gelatin raw material was prepared in 2-fold diluted concentrations of 1.56 to 100 μg/mL with PBS (pH 7.4, Sigma P5368). Samples (typically at 5 mg/mL in PBS starting concentrations) were diluted to 25 to 50 μg/mL in PBS (5 to 10 μL of 0.5 mg/mL protein and 90 to 95 μL PBS). The reactions were carried out in a clear 96-well plate, optimal for UV/Vis absorbance measurements, by mixing 100 μL of sample with 100 μL BCA Working Reagent (25:24:1, Reagent MA:MB:MC) and allowing the plate to rotate gently in a 37 to 39° C. oven for 15 to 30 min to develop the purple color indicating protein content. The absorbance at 562 nm of each reaction was then recorded by a spectrophotometric plate reader (Spectramax M5, Molecular Devices). Each concentration of both samples and standards was done in duplicates and averaged.

Table 7 shows the measured concentrations of fibronectin binding sites, normalized to the protein concentration by comparing the ratio of fibronectin to mg/mL protein, of low numbered and high numbered vials of the composition as prepared in Example 1. The samples were initially melted at 39° C. (for 1 hr) and diluted to 5 mg/mL with PBS. The samples were then melted at 39° C. for 30 min to completely dissolve the gelatin before they were diluted further to 0.5 mg/mL with PBS for both fibronectin binding and BCA protein assays.

TABLE 7

| | Fibronectin Binding/ |
|---|---|
| Vial # | Protein (nmol/mg) |
| #8 | 5.66 |
| #144 | 5.85 |

The test results show that the gelatin raw material demonstrates significant fibronectin binding activity. The fibronectin binding property is retained in the composition of the present disclosure from both a vial collected early during production (vial #8) as well as a vial collected near the end of production (#144).

### Example 6

### Physical Properties of Composition

A dissolution assay was used to measure composition resistance to loss of integrity from solubilization (dissolution) in a physiological solution as a measure of residence time in a wound. This assay was based on standard dissolution testing per the United States Pharmacopeia (USP) XXIII, 1995:1791-1793, modified for the testing of small volumes in a simulated dermal environment. The assay measured the total time (in minutes) for the complete dissolution of a sample disk of the composition at skin temperature 34° C., under a set of defined conditions: one disk per 3 mL PBS (pH 7.4) in a 20-mL vial, rotating gently (200-250 rpm) in a 34° C. oven.

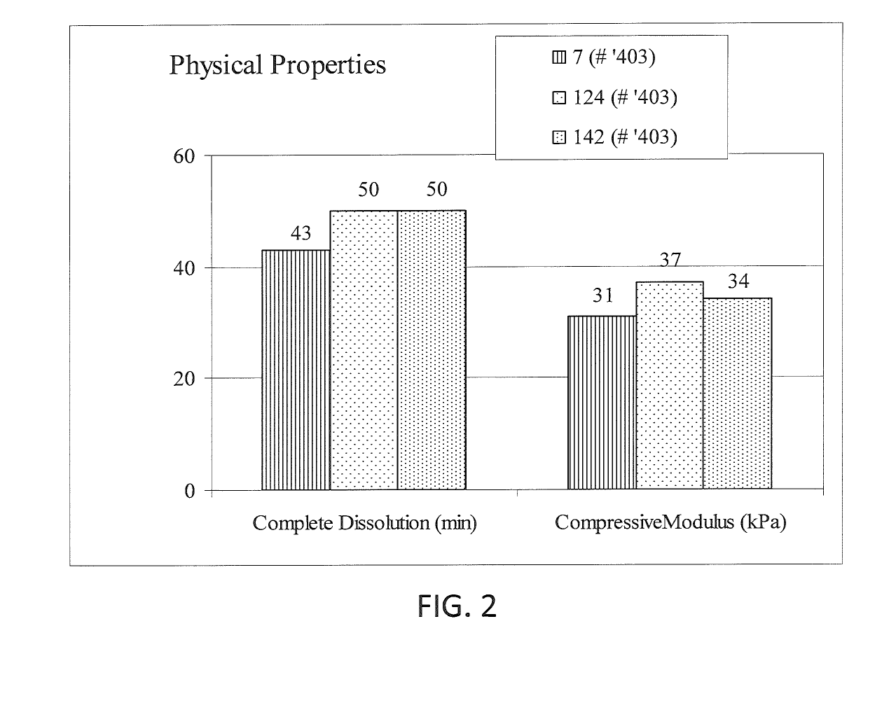

To form the cast disks, vials of the composition from Example 1 were melted at 35° C. for 1.5 hours, mixed by inverting while rotating vials 10-15 times. The melted composition was removed in 1 mL aliquots to fill 6-12 circular wells (disks of 8 mm diameter and 1.5 mm height) of a polypropylene mold, tightly sealed to a flat metal plate by a thin rubber mat. The disks were allowed to cool and form a gel at room temperature for 30 minutes before they were removed from the mold, and stored in pre-weighed capped tubes. The disk weight, typical range of 70-100 mg, was determined for identification. Disks were allowed to set at room temperature for a total of 1-2 hours before they were tested. For each vial of the composition, 3 disks with minimal air bubbles were chosen for the complete dissolution test. Each disk was added to 3 mL PBS, pre-warmed at 34° C., and placed on a rotating platform in 34° C. oven. For each test, the vials were monitored simultaneously by visual inspection, every 5-15 minutes. The samples were observed to be an integral disk that slowly became smaller during the test as the material at the surface was solubilized. The observation of samples was done quickly to minimize the temperature change in these samples. The time for complete dissolution of each disk was recorded (see FIG. 2).

The compressive strength of the composition was also examined by mechanical testing of cylindrical test samples cast from the composition. The testing was based on compressional mechanical testing as described in ASTM D575-91(2007) Test Methods for Rubber Properties in Compression, modified for the testing of small hydrogel samples. To form cast sample cylinders, vials of the composition were melted at 35° C. for 1.5 hours and mixed by inverting while rotating vials 10-15 times. The melted composition was removed in 1-mL aliquots to completely fill, but not over-fill, each circular well (cylinder of 1 cm diameter and 1 cm height) of a 15-well Delrin mold, tightly sealed to a flat metal plate by a thin rubber mat. Typically, 5 cylinders were cast from each vial.

The molds were allowed to set at room temperature for 1.5 hour, and the samples gently removed from the mold. Cylinders were weighed (range=0.7-1 g) for identification, and allowed to set at room temperature for a total of 1-4 hours before they were tested. Each cylinder was then placed in a plastic cylindrical holder (1.5 cm diameter and 1.5 cm height), and was compressed by a flat pin (with head 1 cm diameter and 10 mm height), clamped tightly to the transducer of a mechanical tester (Instron Model 5542). Compressive modulus was measured by the Instron, using a 5 Newton load cell, at a speed of 3 mm/min. The modulus value for compressing each cylinder, calculated by the Instron was reported as the automatic modulus value (in kPa units). For each vial, 3-4 cylinders were measured. For each cylinder, 3-4 consecutive consistent values were averaged. Maximal compressive load (gf) and compressive stress at maximal compressive load (kPa) values were also measured by the Instron instrument. Testing results from vials of composition of Example 1 demonstrate greater physical properties from the high numbered vials produced later during the batch process, as indicated in FIG. 2.

### Example 7

### Stoichiometry of Composition

To examine the suitable formulation stoichiometry, various amounts of gelatin (Gelita USA, Inc., Type A porcine gelatin) and dextran (Sigma Aldrich, Dextran 500,000 MW) were weighed, combined in a 5 mL snap-cap tubes, and 2 mL PBS, pH 7.4 was added. Samples with total polymer weight (gelatin plus dextran) of 200 mg/ml and 230 mg/ml were prepared and tested. The samples were vortex mixed briefly before incubation overnight. All samples were rotated gently at 8 rpm overnight in a 50° C. oven for approximately 19 hours. At that time the samples represented a homogenous, flowable composition. By pouring the compositions into molds used for the dissolution testing, 6-9 disks were cast for each composition, and allowed to set at room temperature for 30 minutes. The de-molded disks (three for each composition) were allowed to set for another 1.5 hours at room temperature. The sample disks were tested in the dissolution assay for physical integrity as described in Example 6 using three disk samples for each test composition, with each disk exposed to 3 mL PBS while gently rotating at 34° C. The disk samples were monitored every 15 minutes for physical integrity.

The dissolution results in Table 8 indicates a lack of physical integrity for samples with 60 weight percent gelatin or less for both polymer concentrations. Samples with greater than 60 weight percent gelatin showed approximately the same amount of dissolution resistance when tested at physiological pH and ionic strength.

TABLE 8

| Gelatin | Dextran | Wt % | Wt % | Dissolution |
|---|---|---|---|---|
| (mg/mL) | (mg/mL) | Gelatin | Dex | Time (min) |
| 120 | 80 | 60% | 40% | 0.000 |
| 140 | 60 | 70% | 30% | 55.000 |
| 160 | 40 | 80% | 20% | 60.000 |
| 180 | 20 | 90% | 10% | 60.000 |
| 200 | 0 | 100% | 0% | 60.000 |
| 138 | 92 | 60% | 40% | 0.000 |
| 161 | 69 | 70% | 30% | 65.000 |
| 184 | 46 | 80% | 20% | 55.000 |
| 207 | 23 | 90% | 10% | 66.667 |
| 230 | 0 | 100% | 0% | 60.000 |

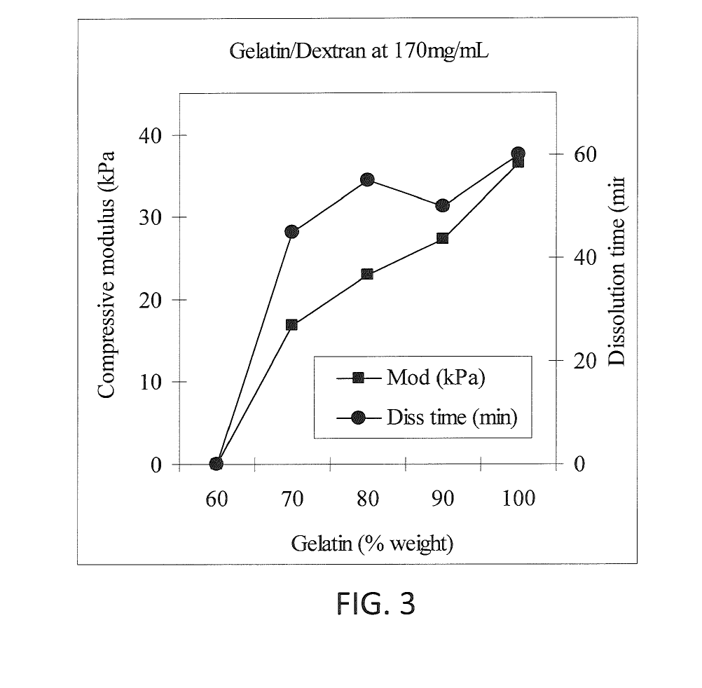

In a second experiment, the gelatin and dextran of the previous experiment were formulated in PBS at a concentration of 170 mg/ml total polymer with 60%, 70%, 80%, 90% and 100% gelatin. The five different ratios of gelatin to dextran formulations were heated to liquefy the material and cast disk samples for characterization of physical properties using the methods of Example 6. The results demonstrated dissolution resistance of the formulations with greater than 60% gelatin, similar to the previous experiment. The compressive testing of the formulations also demonstrated significant increase in physical properties of the formulations with greater than 60% gelatin. The test results are illustrated in FIG. 3.

### Example 8

### Lyophilization of Composition and Reconstitution for Use

The composition of Example 2 was frozen and lyophilized to dryness. The composition was frozen to approximately −30° C. at a cooling rate of approximately 0.05° C./min and held at −30° C. for approximately 12 hours. A vacuum was applied to the frozen composition at −30° C. for approximately 24 hours. Thereafter, the temperature was incrementally increased to approximately −10° C. at a rate of approximately 0.25° C./min. The composition was then held under vacuum at approximately −10° C. for at least 12 hours before the temperature was further increased to approximately 20° C. at a rate of approximately 0.05° C./min. The lyophilized composition was then weighed and placed into vials. Vials containing the lyophilized composition were reconstituted with deionized water to form a flowable composition suitable for administration to a wound. One gram of lyophilized composition was mixed with 5 ml water in a vial to give a final concentration of 0.2 g/ml. The vials were heated at 39° C. for 1 hour and mixed by vortexing at 15 minutes and 30 minutes. The result was a flowable, liquid composition suitable for injection into dermal and subdermal tissues as discussed above.

The lyophilized composition was assayed for fibronectin binding, dissolution time, and compressive modulus using the methods of Examples 5 and 6. The reconstituted composition demonstrated fibronectin binding of 5.13 to 5.88 nmol/mg, dissolution time in the range of 30 to 45 minutes, and compressive modulus in the range of 17 to 29 kPa. When 1% glycerol and 0.1% Tween 20 was added to the reconstitution fluid, the reconstituted composition demonstrated a dissolution time of 33 minutes and a compressive modulus of 34 kPa. When 0.15M NaCl and 1% Tween 20 was added to the reconstitution fluid, the reconstituted composition demonstrated a dissolution time of 30 minutes and a compressive modulus of 33 kPa.

### Example 9

### Reconstitution of Lyophilized Composition for Injection into Wounds/Tissues

The reconstitution of the composition according to Example 8 was examined under various conditions. Vials of the lyophilized composition were mixed with 5 ml of reconstitution fluid preheated to 39° C. to produce a solids content of 0.2 g/ml. The vials were placed in a 39° C. block heater and observed after 5 minutes and vortex mixed. The vials were visually examined subsequently every 1 to 2 minutes and vortex mixed until a flowable, uniform liquid was observed. The reconstituted composition was then tested for ability to flow by injection with a 1 ml syringe through a 25 gauge needle under the conditions described in Example 4.

The liquid composition was considered injectable if 0.5 ml of the fluid was able to be injected. If clogging of the needle during injection of a volume less than 0.5 ml was observed, heating and mixing continued until successful injection. The results are summarized in Table 9, wherein component percentages are provided in weight % unless otherwise stated.

TABLE 9

| | Reconstitution | Time to Reconsitute | Injection Rate at 5N |
|---|---|---|---|
| Vial # | Fluid | (min) | (μL/sec) |
| 1 | Deionized Water | 14 | 21 |
| 2 | Deionized Water, | 13 | 43 |
| | 1% glycerol | | |
| 3 | Deionized Water, | 19 | 18 |
| | 1% Tween 20 | | |
| 4 | Deionized Water, | 9.5 | 33 |
| | 0.15M NaCl, | | |
| | 1% Tween 20 | | |
| 5 | Deionized Water, | 9.3 | 32 |
| | 1% Glycerol, | | |
| | 0.1% Tween 20 | | |
| 6 | Deionized Water, | 12 | 32 |
| | 1% Dextrose, | | |
| | 0.025% Tween 20 | | |

The results demonstrate the ability of a lyophilized composition of the product to be reconstituted into a fluid that is injectable by a fine gauge needle. Reconstitution additives, including hygroscopic excipients, bulking agents, and surfactants, demonstrated the ability to reduce the time to reconstitute the lyophilized composition into an injectable fluid and/or reduce the time to inject the composition under a standard 5 N injection force.

### Example 10

### Tissue Reaction of Composition in Animal Dermal Model

Samples of the composition of Example 1 were heated at 38° C. to form a flowable liquid composition suitable for injection. The composition was injected into the abdominal dermis of forty-four anesthetized adult female Sprague-Dawley rats. The animals were monitored daily until necropsy of four animals each at 1, 3, 6, 9, 12, 15, 18, 21, 24, 27 and 28 days post injection. Tissue samples were harvested at the site of injection and histologically prepared. H&E histopathology of the tissue from the injection sites were examined for cellular reaction and presence of the injected composition.

Histopathology demonstrated the composition to reside in the subdermal space as a single mass of lightly staining material with thin fibrils of eosinophilic fibers uniformly dispersed throughout. The composition showed mild infiltration of polymorphonuclear leukocytes (PMNs) into the composition and surrounding tissue at day 1, with increased amount of cellular infiltration at day 3. The amount of composition appeared decreased at day 3. The 6 day implants demonstrated progressively less of the composition. The 9 day implants showed no composition present in 2 of the animals, with a corresponding decrease in the presence of inflammatory cells. Small amounts of the composition were evident in one animal at tissues from day 12, day 15 and 18. No evidence of the composition was found at day 21 tissues. Small amounts of the composition were found in tissues from two animals at day 24. No evidence of the composition was found in tissues from day 27 and day 28. After the implant material was no longer evident, the injection site tissues demonstrated minimal presence of PMNs, no significant presence of macrophages and no evidence of new collagen deposition or scar formation, demonstrating a very benign tissue reaction. Subsequent histopathology determined that there was no collagen deposition or angiogenesis associated with the composition.

### Example 11

### Administration of Composition of Example 1 to Treat Surgical Wounds

One hundred female subjects between the ages of 18 and 60 years of age undergoing laparotomy or laparoscopy gynecologic procedures were recruited to participate in a prospective randomized, same-scar controlled trial to evaluate the improvement in wound healing by assessing the signs and symptoms of scar formation following a single treatment with the composition of Example 1. Ninety subjects were treated by injection and ten subjects were treated via a catheter.

The subjects' surgical incisions were divided in half, with one half randomly assigned to “treatment” with the composition and the other half assigned to “control” (no treatment) just prior to closing the incision with sutures. Patients returned for follow-up visits through 12 months post-treatment for assessment of the surgical incision halves.

At the time of wound closure in the ninety subjects treated by injection, the composition was heated at 39° C. (+1-2° C.) to form a flowable, injectable liquid. The physician used a syringe and an 18-25 gauge needle to inject the composition into the wound margins. The needle was inserted deep enough into the dermal-subdermal interface and as parallel as possible to the incision wound edge. The needle track and surrounding tissue was infused with sufficient amounts of the composition such that the area surrounding and including the needle track was supplied with approximately 1-2 mL of the composition per 2.5 cm of the wound margin. Effectiveness was evaluated with a validated scar assessment scale using observer (surgeon) and patient assessments of scar characteristics—i.e., the Anchored Visual Analog Scales (AVAS), which were based on the previously validated Visual Analog Scale (VAS), and the Patient and Observer Scar Assessment Scale (POSAS).

A standard VAS evaluation uses a 100 mm horizontal visual analog cosmetic scale marked “worst possible scar” at the left end and “best possible scar” at the right end. The evaluator was asked to mark along the horizontal scale indicating the overall aesthetic appearance of the scar. Numeric scores were calculated by measuring horizontal distance from the low end of the scale to the evaluator's mark and rounding to the nearest millimeter. The AVAS scale uses a horizontal visual analog cosmetic scale that allows the evaluator to select the worst scar half as an anchor for the left end of the scale while using normal skin at the right end of the scale. The evaluator was asked to mark along the horizontal scale indicating the overall aesthetic appearance of the better scar half between the two ends. Numeric scores were calculated by measuring horizontal distance from the low end of the scale to the evaluator's mark and rounding to the nearest millimeter.

The observer and patient AVAS scores through 12 months were analyzed using generalized estimating equations (GEE), which is a method for analyzing longitudinal data that takes into account the expected correlation among observations for the same subject. The AVAS analyses were performed assuming an autoregressive structure. The GEE analyses were used to obtain an estimated mean difference between treatments (control side-treated side) overall and for the individual characteristics for the AVAS scores, and to perform a two-sided normal approximation test for statistical significance.

The ninety subjects treated by injection of the composition were evaluated by a surgeon observer and the treated subject using the anchored visual analog scale (AVAS). After treatment of the first 30 subjects, the protocol was amended to include the AVAS scale. For the first 30 subjects enrolled, the AVAS scores were only collected from the observer and patient at the 9 and 12 month follow-up visits. The next 60 subjects enrolled had AVAS data collected from the observer and patient at all follow-up time points. When evaluating AVAS for treated versus control, the overall estimated AVAS difference for the Observer and Patient evaluation through 12 months were 7.49 mm (p=0.0018) and 9.86 mm (p=0.0009), respectively, in favor of the treated side. The results for the Observer and Patient AVAS are presented in Table 10.

TABLE 10
Summary of treatment effect estimate and P-value by GEE (Observer
and Patient AVAS Scores) through 12 months (n = 90)

| Parameter | Estimate | p-value |
|---|---|---|
| Observer AVAS | 7.4939 | 0.0018 |
| Patient AVAS | 9.8605 | 0.0009 |

The effectiveness of the composition was also evaluated with the validated POSAS scar assessment scale by GEE, as noted above, using observer (physician/investigator) and patient live assessments of scar characteristics. The observer scale of the POSAS consists of five wound healing characteristics which are scored numerically on a scale of 1-10, whereby 1 is the best score and 10 is the worst. The total score of the observer scale consists of adding the scores of each of the five items (range, 5 to 50). The lowest score (5) reflects normal skin. The results are shown below in Table 11 and Table 12. When evaluating Observer POSAS scores for treated versus control, the overall estimated difference through 12 months was 0.98 (p=0.0051), in favor of the treated side and the estimated POSAS differences for the individual Observer-evaluated characteristics ranged from 0.20 to 0.26 and were all significantly in favor of the treated scar half. When evaluating Patient POSAS scores for treated versus control, the overall estimated difference through 12 months was 1.19 (p=0.0012) in favor of the treated side and the estimated POSAS differences for the individual Patient-evaluated characteristics ranged from 0.09 to 0.36 and were all significantly in favor of the treated scar half

TABLE 11
Observer POSAS Score Through 12 months (n = 90)

| Characteristic | Estimate | p-value |
|---|---|---|
| Vascularization | 0.2598 | 0.0005 |
| Pigmentation | 0.2453 | 0.0018 |
| Thickness | 0.1996 | 0.0042 |
| Relief | 0.2275 | <0.0001 |
| Pliability | 0.2209 | <0.0001 |
| Overall | 0.9839 | 0.0051 |

TABLE 12
Subject POSAS Score Through 12 months (n = 90)

| Parameter | Estimate | p-value |
|---|---|---|
| Pain | 0.0918 | 0.0375 |
| Itching | 0.1369 | 0.0248 |
| Color | 0.2700 | 0.0018 |
| Stiffness | 0.3581 | <0.0001 |
| Thickness | 0.3261 | 0.0002 |
| Irregularity | 0.1612 | 0.0126 |
| Overall | 1.1905 | 0.0012 |

### Example 12

### Effect of Composition of Example 1 for Surgical Revision of Keloids

A study was performed to evaluate the use of the composition in the reduction of the volume, appearance, and/or symptoms associated with keloid scarring in subjects undergoing surgical revision of keloid scars by excision of the keloid tissue as compared to recurrence rates reported in contemporary literature.

Nineteen subjects with 26 ear keloids were enrolled in the study. Keloids on the ears of the study subjects were surgically removed and the wound margins of the incision were identified. The composition of Example 1 was heated at 39° C. (+/−2° C.) for a minimum of 60 minutes prior to injection. A 25 gauge needle was inserted deep enough into tissue to be in the dermal/subdermal interface and as parallel as possible to the incision wound edge. The investigator injected the composition such that it was dispersed into the infusion track and into the surrounding tissue as the needle was withdrawn. The needle track and surrounding tissue in the wound margins were infused with sufficient amounts of composition, an average of 1.48 ml per every 2.5 cm increment of the incision length on each side of the wound margin. The investigator was also guided by visual and tactile feedback during the injection process such as skin tension caused by the injection, ease of flow of the composition from the needle, blanching of the tissue, and the physical ability of the tissue to contain the composition. All incision locations (i.e., right ear, left ear and/or both ears) were closed in the same manner.

A protocol for keloid volume measurement using impressions with dental alginate was used to measure lesion size volume. The volume of the keloid was measured by filling the alginate impression with water and weighing the volume on a calibrated scale. This allowed a numerical value to be obtained for the volume of the lesion. Impressions were to be made of only those lesions amenable to alginate impressions. An alginate impression was made and volumes were calculated for each subject at baseline. At the 12 month follow-up visits, five ears (19.2%) were identified as having recurrences based on clinical examination. Molds were cast for each ear. The pre-surgical volumes and the 12 month volumes of the molds are included in Table 13 below. Additionally, the percentage of the pre-surgical volume has been calculated for each of the 12 month molds. Of the five recurrences from treatment with the composition, two of the recurrences demonstrated clinically significant lesion size of 5% or greater at 12 months.

TABLE 13
Lesion Volume as Measured by Alginate Mold

| | Pre-surgical | 12 Month | % of Pre- |
|---|---|---|---|
| Subject | Volume | Volume | surgical Volume |
| 08-02-TS | 3.867 gm | 0.18 gm | 4.7% |
| 08-09-KA-R | 7.641 gm | 0.040 gm | 0.5% |
| 08-11-TO-R | 2.542 gm | 0.365 gm | 14.4% |
| 08-11-TO-L | 2.664 gm | 0.345 gm | 13.0% |
| 08-18-DS | 3.138 gm | 0.027 gm | 0.9% |

The literature specific to keloid recurrence following surgical excision indicates that concomitant therapy (e.g., surgical excision/corticosteroid injections) has become the standard of care. Included in Table 14 below is the literature most relevant to keloid recurrence following surgical excision alone.

TABLE 14

| Study | Year | Excision | # Patients | % of Recurrence |
|---|---|---|---|---|
| Nason | 1942 | Scalpel | 12 | 83% |
| Arnold | 1959 | Scalpel | 14 | 86% |
| Conway | 1960 | Scalpel | 28 | 45% |
| Cosman | 1961 | Scalpel | 25 | 54% |
| Cosman | 1972 | Scalpel | 7 | 57% |
| Cosman | 1974 | Scalpel | 20 | 73% |
| Ramakrishnan | 1974 | Scalpel | 108 | 80% |
| Oluwasanmi | 1974 | Scalpel | 41 | 93% |
| Apfelberg | 1989 | Laser | 9 | 89% |
| Stern | 1989 | Laser | 23 | 74% |
| Berman | 1997 | Scalpel | 43 | 51% |
| Kim | 2004 | Scalpel | 9 | 44% |

Recurrence was documented in the literature between 44-93%. A study by Berman et al. (Berman B., Flores F. “Recurrence rates of excised keloids treated with postoperative triamcinolone acetonide injections or interferon alfa-2b injections”, J AM Acad Dermatol. 1997; 37:755-7) has been deemed to be the best contemporary literature comparator, as the publication includes a data set of a significant number of subjects. When considering surgical excision of earlobe keloids specifically, Berman et al. studied 43 patients undergoing surgical excision alone (86% with earlobe or ear helix keloids), 51.2% of the keloids recurred in an average of 6.5 months. Analyses were performed comparing the treatment results using the composition to results from Berman et al. (1997).

Using a 2×2 contingency table, the treatment results using the present composition (as described above) were compared to the Berman et al. publication. Analysis using Fisher's exact test demonstrated a two tailed p value of 0.011, indicating a statistically significant difference. The composition was found to be significantly superior to reported surgical excision alone for preventing keloid recurrence when evaluated at 12 months. The composition demonstrated the ability to prevent and minimize keloid recurrence when administered by injection with a small gauge needle to dermal and subdermal tissues during keloid revision surgery.

### Example 13

### Stability Comparison of Compositions of Examples 1 and 2

A comparative ultrasonic analysis was carried out using the gelatin/dextran compositions prepared as described in Example 1 and Example 2. Previously prepared test samples were stored at 2-8° C. until testing. Glass vials containing solid gel samples were placed into a water bath at 40° C. to melt the gel samples. Melted gel samples were quickly degassed in plastic syringes using a centrifuge at 3,000 rpm and loaded into the sample cell of an HR-US 102 ultrasonic spectrometer pre-equilibrated at 40° C. A reference ultrasonic cell was filled with deionized, degassed water. Both cells were closed tightly using screw type caps with silicone/PTF septa. Comparison of ultrasonic profiles (ultrasonic velocity and attenuation) of the melted (i.e., flowable liquid) compositions were measured continuously at a frequency range of 2-12 MHz with the following temperature ramp: i) held at 40° C. for 40 minutes; ii) cooled to 35° C. over a time of 10 minutes; iii) held at 35° C. for 700 minutes+. The ultrasonic velocity and attenuation in samples were measured continuously in kinetics mode.

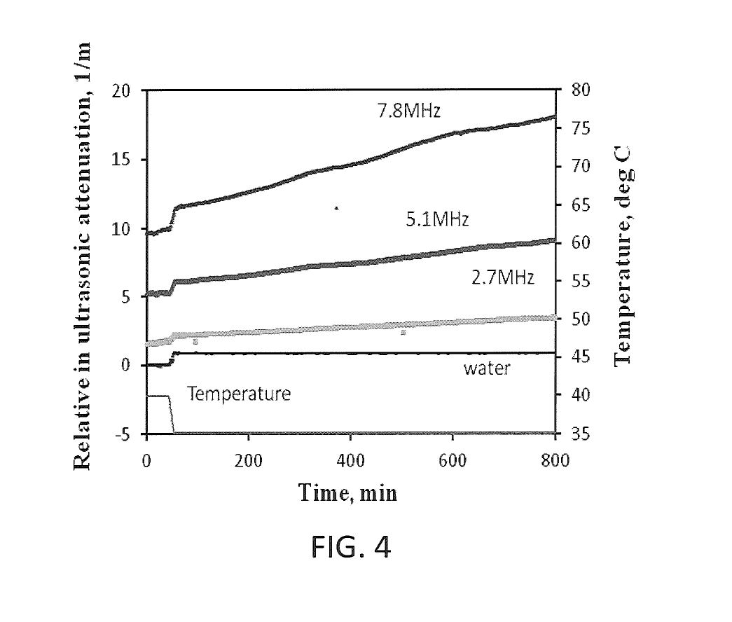

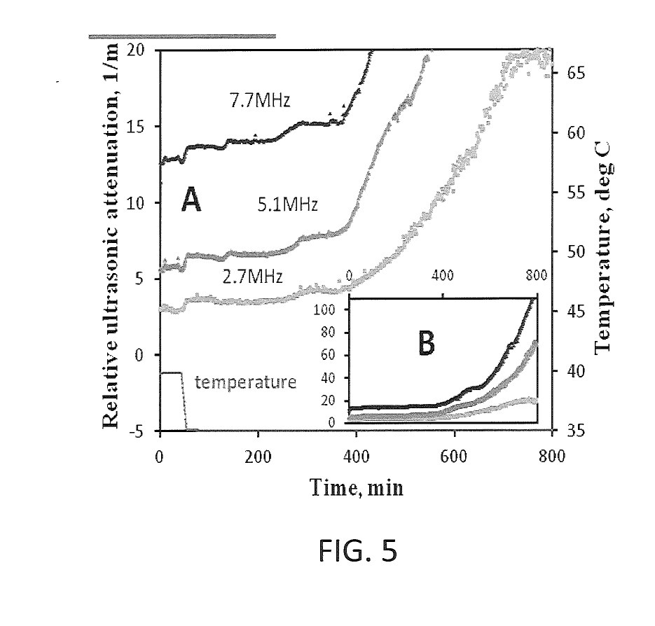

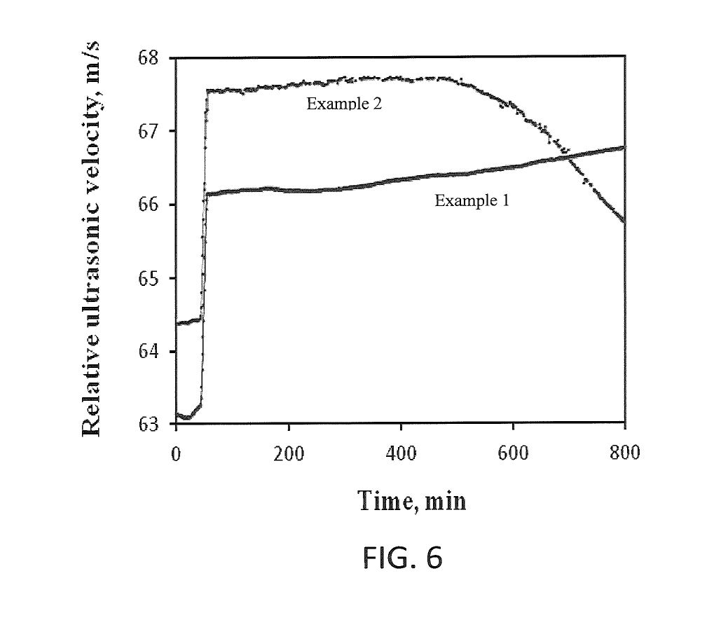

The ultrasonic evaluation revealed significantly and surprisingly different behavior of the Example 1 and Example 2 samples in their temperature and time profiling. This is shown in FIGS. 4 through 6, which illustrate the evolution of relative ultrasonic attenuation (FIG. 4 for Example 1 Composition and FIG. 5 for Example 2 Composition) and ultrasonic velocity (FIG. 6) in gel samples with time, upon quick cooling from 40° C. to 35° C. and following long term isothermal regime at 35° C.

Initially, it can be seen in FIGS. 4 through 6 that the Example 2 sample had a higher ultrasonic attenuation and ultrasonic velocity at 40° C. compared to the Example 1 sample. Both samples exhibited a sharp increase in ultrasonic attenuation and ultrasonic velocity associated with the rapid cooling from 40° C. to 35° C.; however, the Example 2 sample did show a measurably greater increase than the Example 1 sample. The difference in starting ultrasonic attenuation and ultrasonic velocity values showed that the as-formed hydrogels already exhibited different polymeric structural/conformational states. The difference in ultrasonic attenuation particularly showed that the Example 1 samples and the Example 2 samples were exhibiting different relaxation dynamics at ultrasonic frequencies.

The differences in the hydrogel stability of the samples became more apparent as the test continued with a hold at 35° C. As seen in FIG. 4 and FIG. 6, the Example 1 sample showed a small but continuous increase in ultrasonic attenuation and ultrasonic velocity with time. This showed that although hydrogel restructuring was occurring (which is desirable to aid in eventual physiological resorption of the hydrogel components), the restructuring process in the Example 1 hydrogel was mild. As further seen in Table 15, Example 1 hydrogels exhibited stability in excess of the test duration. The Example 2 sample showed a similar profile within the first 400 minutes of testing. Thereafter, however, the Example 2 samples exhibited a rapid, significant increase in ultrasonic attenuation (FIG. 5) and decrease in ultrasonic velocity (FIG. 6), which was indicative of extensive restructuring of the Example 2 hydrogel. Although not wishing to be bound by theory, it is believed that this restructuring corresponds to a phase separation of the gelatin and the polymeric carbohydrate (dextran in these examples)—i.e., a loss of the ionic and hydrogen bonding between the respective polymers that maintain the hydrogel structure. The effect of processing of the hydrogel on its stability is also evident in that the varying salt content of the Example 2 samples (i.e., formed using PBS with high phosphate ion concentration) is believed to lead to dehydration of the hydrogel, which also can correspond to the structural rearrangement of the polymers. Table 15 provides restructuring transition times for two samples of Example 1 hydrogel (N1 and N2) and two samples of Example 2 hydrogel (N3 and N4), which further illustrate the differences in the structural stability of the samples. The samples from Example 2 showed a significantly decreased hydrogel stability in transitioning out of the hydrogel state as compared to the samples from Example 1 (N1 and N2).

TABLE 15

| Sample (N) | Composition | Transition Time (min) |
|---|---|---|
| 1 | Example 1 | 950 (+/−50) |
| 2 | Example 1 | >1,500 |
| 3 | Example 2 | 750 (+/−20) |
| 4 | Example 2 | 590 (+/−50) |

### Example 14

### Effect of Composition of Example 2 for Surgical Revision of Scars

A similar study to that described above in Example 11 and Example 12 was performed using the composition of Example 2. This study was a prospective, randomized, single blind, same scar, placebo-controlled feasibility study evaluating the safety and effectiveness of the composition prepared as described in Example 2 in improving the appearance and reducing the signs, symptoms, and recurrence of scarring in subjects undergoing surgical revision of scars. The test encompassed fourteen patients electing to undergo scar revision procedures at the participating centers. During the scar revision procedure, the incision was divided in half and each side was randomized to either the Example 2 composition or placebo (saline) injection. Post-operative care was provided as per the standard-of-care and the surgeon's normal routine, except as excluded in the procedure section of the protocol.

As in Example 11, the effectiveness of the composition of Example 2 was also evaluated using the Patient and Observer Scar Assessment Scale (POSAS). The POSAS evaluation incorporated patient and observer assessments, where the observer assessment was based on direct observation of the patient. The patient evaluated wound healing characteristics of pain, itching, color, stiffness, thickness, and irregularity, each on a 10-point scale where “1” is best and “10” is worst. The observer evaluated vascularization, pigmentation, thickness, relief, and pliability using the same ten point scale. The individual scores were summed for the patient and the observer separately, with higher scores representing a worse result and lower scores representing scars more closely resembling normal skin. The total score average difference (control minus treated) is presented in Table 16. A negative number represents the control side being assessed as better than the treated side.

TABLE 16

| | | | | | | |
|---|---|---|---|---|---|---|
| Composition | Sample* | 1M | 3M | 6M | 9M | 12M |

Patient POSAS

| | | | | | | |
|---|---|---|---|---|---|---|
| Example 1 as | N = 90 | 0.91 | 0.95 | 1.06 | 2.01 | 0.54 |
| used in Example | | | | | | |
| 11 Study | | | | | | |
| Example 2 | N = 14 | −0.43 | 0.21 | −4.09 | −4.27 | −0.86 |

Observer POSAS

| | | | | | | |
|---|---|---|---|---|---|---|
| Example 1 as | N = 90 | 1.25 | 1.56 | 0.88 | 1.34 | 1.29 |
| used in Example | | | | | | |
| 11 Study | | | | | | |
| Example 2 | N = 14 | −5.79 | −1.64 | −4.18 | −4.55 | −1.43 |

*not all patients returned for all follow-up visits

As can be seen from Table 16, the POSAS evaluations indicated that the hydrogel of Example 1 provided a significantly better result in scar revision as compared to the hydrogel of Example 2. It was surprising to find that not only did the method of manufacture of the hydrogel significantly impact the stability of the hydrogel but that it also significantly impacted the clinical efficacy of the hydrogel in the dermal wound models evaluated. It was particularly unexpected to find that the Example 2 hydrogel was mainly evaluated as performing worse than placebo while all Example 1 hydrogel evaluations were very good and in every case performed better than placebo.

### Example 15

### Phase Separation Study of Previously Prepared and Gelled Compositions

A study was performed to evaluate the stability of hydrogel compositions prepared with different cooling protocols. Testing was carried out on four different lots of hydrogel compositions according to the formula of Example 1. In each case, the hydrogel was formed in the liquid state, cooled to gelation, and stored in refrigeration at 2-8° C. The hydrogels of Lot 0017-4 and 0017-188 were prepared as described herein by mixing at 50° C. to form the liquid hydrogel, cooling to a holding temperature below 50° C. and above the transition temperature from liquid to solid or semi-solid, and then further cooling the composition below the transition temperature. The hydrogels of Lot FG-13-0049 and FG-13-0075 were also prepared by mixing at 50° C. to form the liquid hydrogel but were maintained at 50° C. until cooling directly to below the transition temperature.

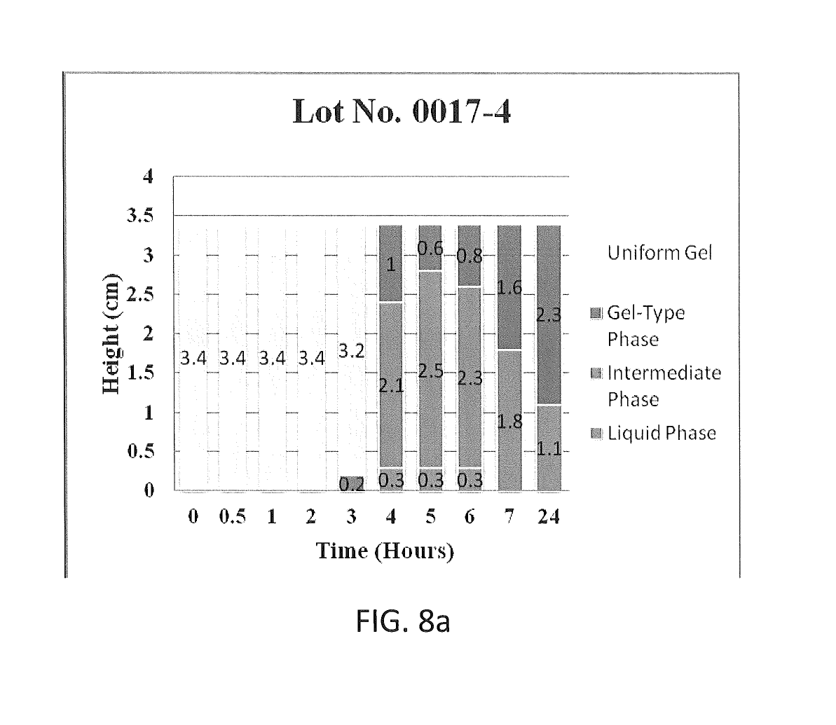

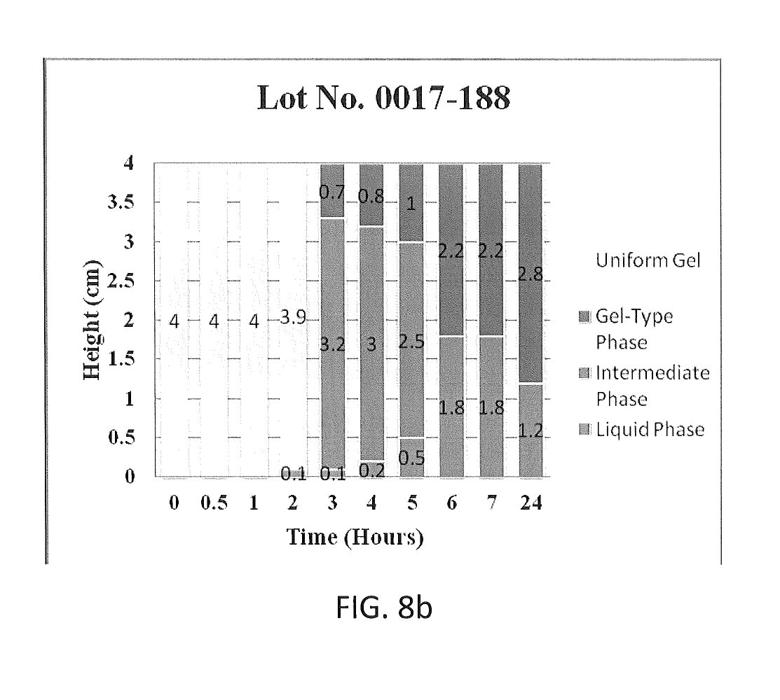

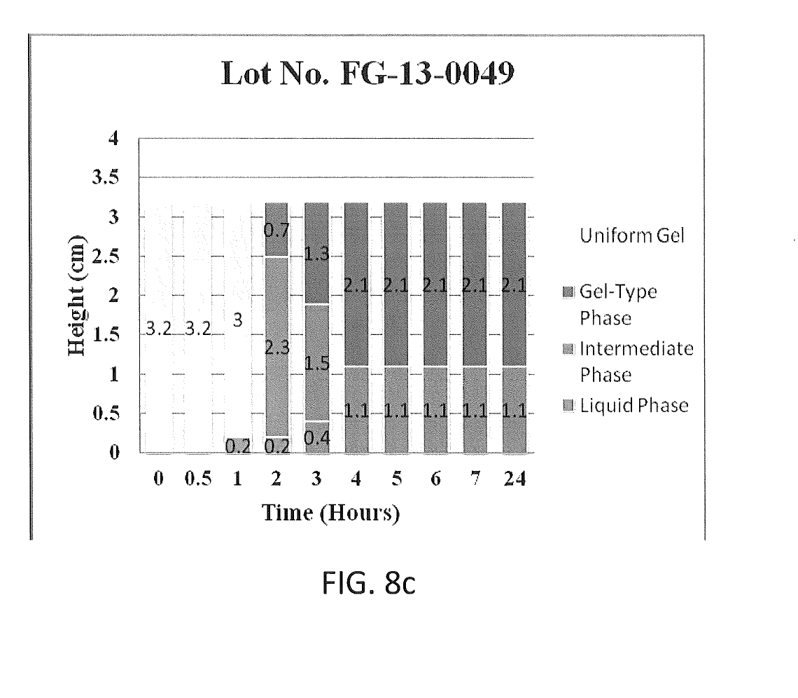

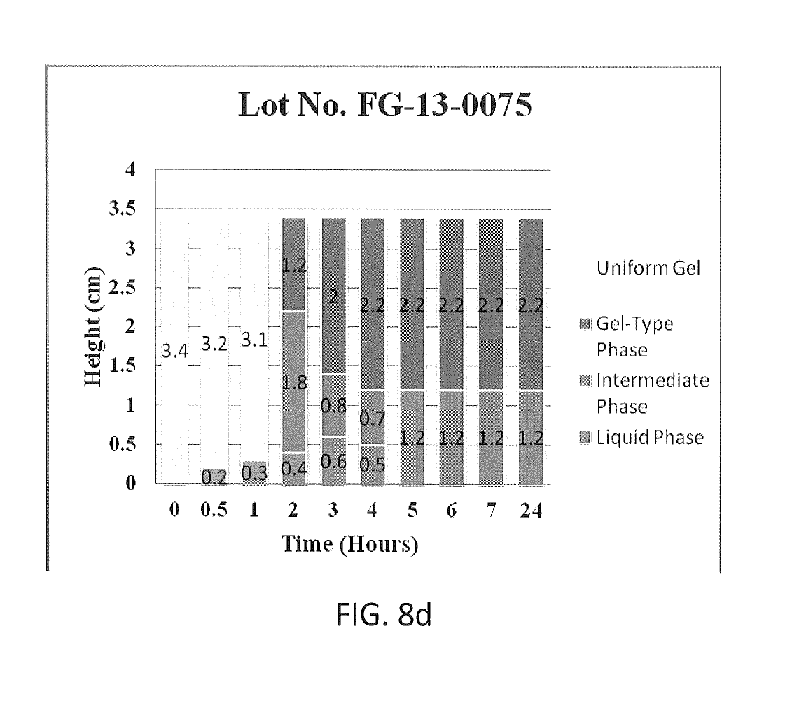

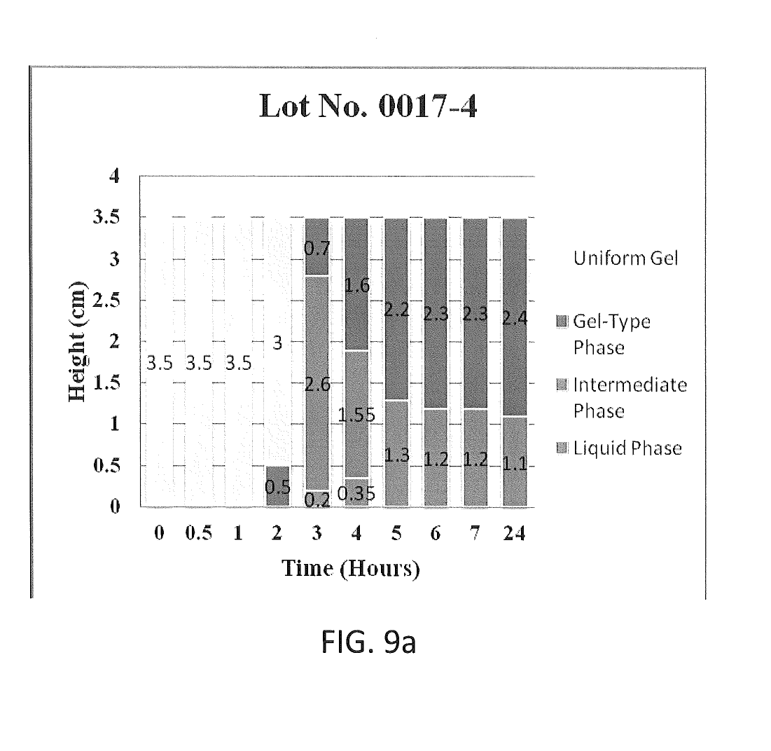

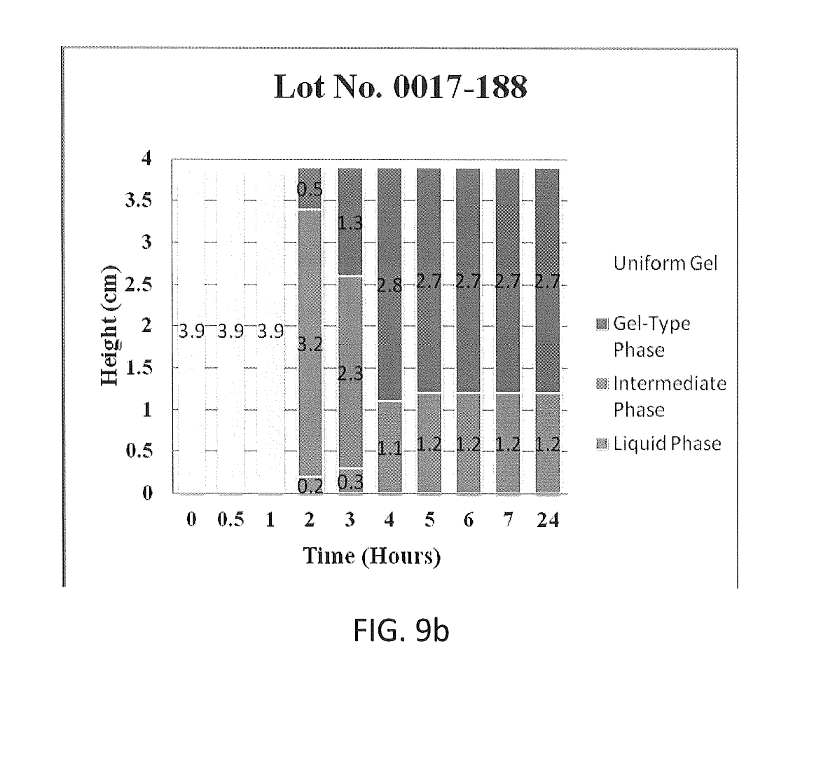

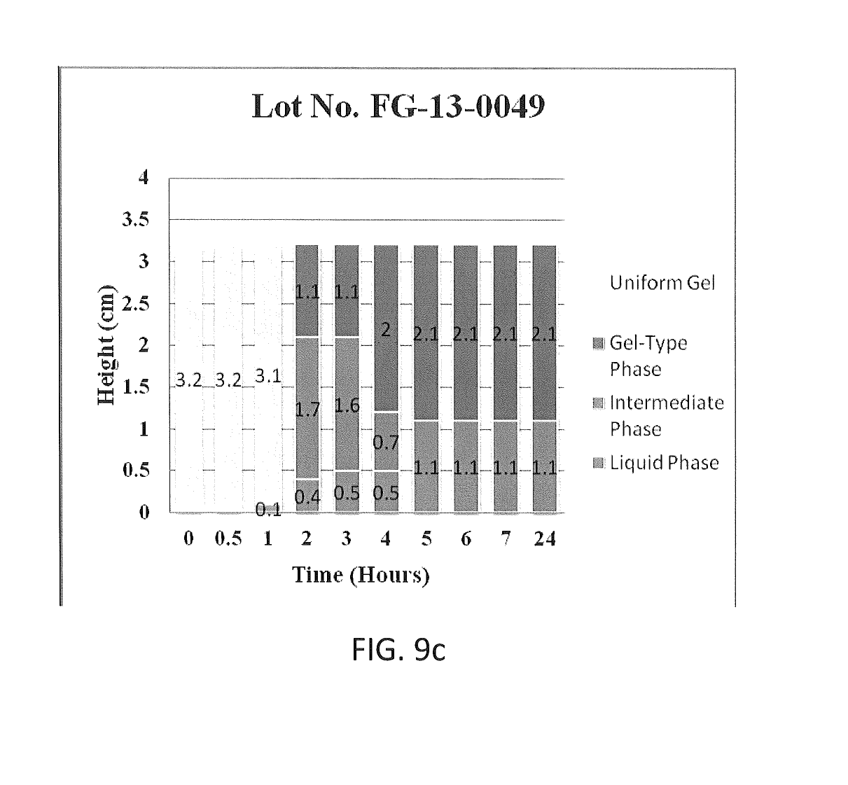

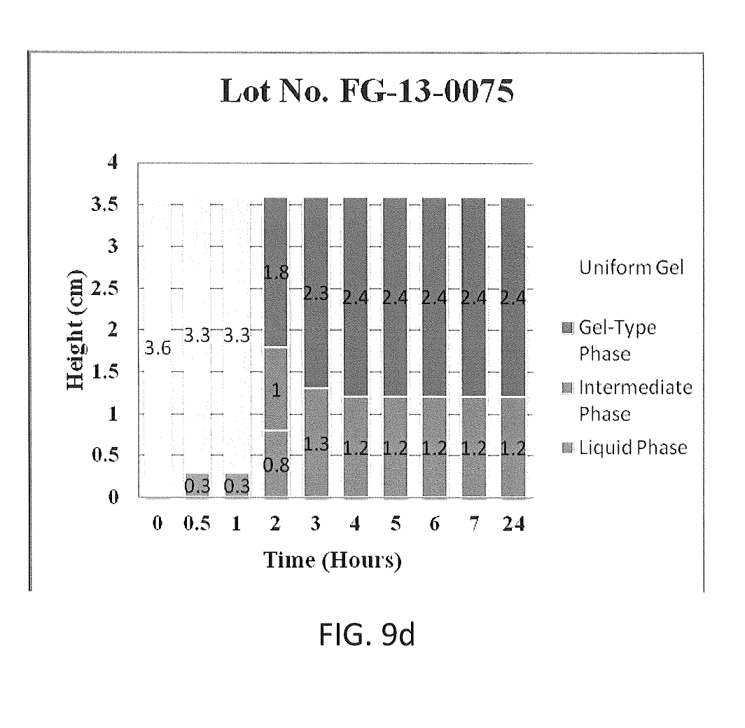

For testing, two water baths were equilibrated for a minimum of 60 minutes at temperatures of 40° C. and 50° C. A vial from each of the four lots was removed from storage at 2-8° C. and placed into the 40° C. water bath. A vial from each of the four lots also was removed from storage at 2-8° C. and placed into the 50° C. water bath. Phase separation of the samples at the two different temperatures was monitored for 7 hours and allowed to incubate for approximately 24 hours at the designated temperatures. A summary of the phase separation observed for the vials at 40° C. and 50° C. is provided in Tables 17 and 18, respectively. The test results are graphically illustrated in FIGS. 8a-d and FIGS. 9a-d, respectively.

TABLE 17
Phase Separation at 40° C.

| | Lot | Lot | Lot | Lot |
|---|---|---|---|---|
| Test Point | 0017-4 | 0017-188 | FG-13-0049 | FG-13-0075 |
| First Sign of Phase | 3 hrs | 2 hrs | 1 hr | 0.5 hr |
| Separation | | | | |
| First Sign of Three | 4 hrs | 3 hrs | 2 hrs | 2 hrs |
| Phases | | | | |
| Distinct Two Phase | 7 hrs | 6 hrs | 4 hrs | 5 hrs |
| Formation | | | | |

TABLE 18
Phase Separation at 50° C.

| | Lot | Lot | Lot | Lot |
|---|---|---|---|---|
| Test Point | 0017-4 | 0017-188 | FG-13-0049 | FG-13-0075 |
| First Sign of Phase | 2 hrs | 2 hrs | 1 hr | 0.5 hr |
| Separation | | | | |
| First Sign of Three | 3 hrs | 2 hrs | 2 hrs | 2 hrs |
| Phases | | | | |
| Distinct Two Phase | 5 hrs | 4 hrs | 5 hrs | 3 hrs |
| Formation | | | | |

As can be seen from the above tables and the noted figures, the compositions prepared by including an intermediate cooling step to a temperature below the initial mixing temperature but above the phase transition temperature maintained single phase integrity at least twice as long as compositions prepared without the intermediate cooling step and where the composition was maintained at the higher temperature for a longer period of time. This indicates that the compositions prepared as described herein with the intermediate cooling step exhibit greater stability of the gel structure and resistance against phase separation under treatment conditions.

### Example 16

### Phase Separation Study of Previously Prepared and Gelled Compositions

A further study was performed to evaluate the use of the composition of the present disclosure in the reduction of the volume, appearance, and/or symptoms associated with keloid scarring in subjects undergoing surgical revision of keloid scars by excision of the keloid tissue.

Subjects with a total of 65 ear keloids were enrolled in the study. Removal of the keloid and treatment of the surgical site was carried out using the protocol described above in Example 12. The amount of the hydrogel composition injected at the surgical sites averaged 2.8 ml (ranging from a low of 0.4 ml to a high of 9.5 ml depending upon the size of the surgical site). The average amount of the hydrogel composition injected per 2.5 cm of incision was 1.29 ml (ranging from a low of 0.5 ml/cm to a high of 1.6 ml/cm).

The protocol for keloid volume measurement using impressions with dental alginate as described in Example 12 was used in the present testing to measure lesion size volume. The pre-surgical volumes and the follow-up volumes of the molds are included in Table 19 below. The primary effectiveness endpoint of the present testing was based on the proportion of patients with recurrence of keloid scar post excision and was defined as the presence of scar tissue volume of greater than 0.3 cm³ (0.3 grams). Effectiveness also was evaluated based on the Observer and Patient assessed POSAS scores, which are provided in Table 20 and Table 21, respectively.

Referring to Table 19, of the original 65 keloids treated, only nine instances exhibited measurable lesions following the surgical procedure (evaluated at one month, three months, and six months post treatment). As can be seen in Table 19, none of the measurable lesion volumes at six months met the criteria of keloid recurrence (i.e., presence of scar tissue volume of greater than 0.3 cm³. Thus, the 6 month recurrence rate in this study as defined by the protocol as 0% and was demonstrated to be statistically superior to the scientific literature when evaluating keloid recurrence rates following surgical excision (see Table 14 and associated discussion above). In Table 19, Subject 01-20-PM exhibited overall swelling of the ear of unknown cause at the 3 month visit, which accounted for the measured volume. The swelling of unknown cause resolved without treatment and, at the 6 month evaluation, no measurable lesion was present.

TABLE 19

| | Pre-Surgical | | 1 Month | 3 Month | 6 Month |
|---|---|---|---|---|---|
| Subject | Volume | | Volume | Volume | Volume |
| 01-01-MB-L | 0.519 | g | N/A | N/A | 0.096 g |
| 01-04-KH | 3.87 | g | N/A | N/A | 0.042 g |
| 01-05-JB | 4.4 | g | N/A | 0.084 g | 0.198 g |
| 01-11-ML | 0.478 | g | N/A | N/A | 0.083 g |
| 01-13-LM | 3.91 | g | N/A | 0.08 g | N/A |
| 01-20-PM | 0.467 | g | N/A | 0.564 g | N/A |
| 08-01-OW | 0.858 | g | N/A | N/A | 0.012 g |
| 11-01-AS | 1.378 | g | N/A | N/A | 0.082 g |
| 11-09-LT | 0.958 | g | N/A | 0.012 g | 0.183 g |

Referring to Table 20, pain, itching, color difference, stiffness, thickness, irregularity, and overall opinion were considered by the subjects. Each category was scored on a scale of 1-10, wherein 1 is best and 10 is worst. The average scores are shown in Table 20. As can be seen, the subjects on average rated the treated sites to be significantly improved after the treatment.

Referring to Table 21, vascularity, pigmentation, thickness, relief, pliability, surface area, and overall opinion were considered by the respective investigators. Each category was scored on a scale of 1-10 wherein 1 is best and 10 is worst. The average scores are shown in Table 21. As can be seen, the investigators on average rated the treated sites to be significantly improved after treatment.

TABLE 20
Subject POSAS Assessment Scores

| | Baseline | 1 Month | 3 Months | 6 Months |
|---|---|---|---|---|
| Parameter | N-65 | N = 60 | N = 61 | N = 59 |
| Pain | 3.0 | 1.8 | 1.7 | 1.6 |
| Itching | 3.9 | 2.2 | 2.4 | 2.2 |
| Color | 4.4 | 2.6 | 2.5 | 2.3 |
| Stiffness | 6.1 | 2.8 | 3.1 | 2.7 |
| Thickness | 7.5 | 2.6 | 2.8 | 2.9 |
| Irregularity | 7.8 | 2.5 | 2.8 | 2.8 |
| Overall | 7.7 | 2.4 | 2.7 | 2.8 |

TABLE 21
Investigator POSAS Assessment Scores

| | Baseline | 1 Month | 3 Months | 6 Months |
|---|---|---|---|---|
| Parameter | N-65 | N = 60 | N = 61 | N = 59 |
| Vascularity | 5.3 | 2.6 | 2.6 | 2.4 |
| Pigmentation | 5.7 | 2.7 | 2.6 | 2.4 |
| Thickness | 7.4 | 2.6 | 3.0 | 2.9 |
| Relief | 7.0 | 2.5 | 2.9 | 2.8 |
| Pliability | 7.3 | 2.7 | 2.9 | 2.9 |
| Surface Area | 7.6 | 2.5 | 2.9 | 2.7 |
| Overall Opinion | 7.2 | 2.7 | 2.8 | 2.8 |

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed herein and that modifications and other embodiments are intended to be included within the scope of the appended claims. Although specific terms are employed herein, they are used in a generic and descriptive sense only and not for purposes of limitation.

1. A method of preparing a stable hydrogel composition, the method comprising:
mixing gelatin and a polymeric carbohydrate in an aqueous medium at a first temperature to form a liquid hydrogel composition wherein the gelatin comprises about 60% by weight or greater of the total weight of the combination of the gelatin and the polymeric carbohydrate present in the composition;
cooling the liquid hydrogel composition with constant mixing to a holding temperature that is at least about 5° C. less than the first temperature and greater than the gelation temperature at which the liquid hydrogel composition transitions to a solid or semi-solid hydrogel composition, the holding temperature being within about 7° C. of the gelation temperature; and
further cooling the liquid hydrogel composition to the gelation temperature to transition the liquid hydrogel composition to a stable, solid or semi-solid hydrogel composition.

2. The method according to claim 1, wherein the stable hydrogel composition exhibits an ultrasonic attenuation (U_{A}) hydrogel stability factor of 0.4 for at least 500 minutes when tested at a temperature of 35° C. and a frequency of 2-8 MHz, or wherein the stable hydrogel composition exhibits a U_{A} hydrogel stability factor of 0.5 for at least 600 minutes when tested at a temperature of 35° C. and a frequency of 2-8 MHz.

3. The method according to claim 1, wherein the stable hydrogel composition is defined by one or more of: a U_{A} hydrogel stability factor of 0.3 for at least 500 minutes when tested at a temperature of 35° C. and a frequency of 2.7 MHz; a U_{A} hydrogel stability factor of 0.4 for at least 600 minutes when tested at a temperature of 35° C. and a frequency of 2.7 MHz; a U_{A} hydrogel stability factor of 0.2 for at least 500 minutes when tested at a temperature of 35° C. and a frequency of 5.1 MHz; a U_{A} hydrogel stability factor of 0.3 for at least 600 minutes when tested at a temperature of 35° C. and a frequency of 5.1 MHz; a U_{A} hydrogel stability factor of 0.35 for at least 500 minutes when tested at a temperature of 35° C. and a frequency of 7.8 MHz; and a U_{A} hydrogel stability factor of 0.5 for at least 600 minutes when tested at a temperature of 35° C. and a frequency of 7.8 MHz.

4. The method according to claim 1, wherein the first temperature is about 45° C. or greater.

5. The method according to claim 1, wherein the gelation temperature is about 35° C.

6. The method according to claim 1, wherein further cooling to the gelation temperature is carried out in a time of less than about 2 hours.

7. The method according to claim 1, wherein the further cooling comprises cooling the stable, solid or semi-solid hydrogel composition to a storage temperature of about 1° C. to about 12° C.

8. The method according to claim 1, wherein the aqueous medium has an osmolality of less than about 400 mOsm/kg.

9. The method according to claim 1, wherein the aqueous medium has an osmolality of about 25 mOsm/kg to about 375 mOsm/kg.

10. The method according to claim 1, wherein the aqueous medium has one or both of a phosphate ion concentration of no greater than about 20 mM and a carbonate ion concentration of no greater than about 20 mM.

11. The method according to claim 1, wherein the aqueous medium comprises Medium 199.

12. The method according to claim 1, wherein the total concentration of the gelatin and the polymeric carbohydrate in the stable hydrogel composition is about 50 mg/mL to about 400 mg/mL.

13. The method according to claim 1, further comprising lyophilizing the stable hydrogel composition.

14. A stable hydrogel composition prepared according to the method of claim 1, wherein the stable hydrogel composition exhibits one or more of the following:
an ultrasonic attenuation (U_{A}) hydrogel stability factor of 0.4 for at least 500 minutes when tested at a temperature of 35° C. and a frequency of 2-8 MHz;
a U_{A} hydrogel stability factor of 0.5 for at least 600 minutes when tested at a temperature of 35° C. and a frequency of 2-8 MHz;
a flow rate of about 10 μL/s or greater when forced from a syringe through a ⅝ inch long 25 gauge needle under a syringe plunger pressure of 5 N at a temperature of 35° C. to 39° C.;
a viscosity of about 1.5 Pa-s or less at a temperature of 35° C. to 39° C.;
a fibronectin binding activity of about 3 nmol/mg or greater; and
a residence time in the dermal or subdermal tissue of a mammal of about 3 days or greater.

15. A flowable, injectable composition comprising gelatin and a polymeric carbohydrate, the composition being a hydrogel reconstituted from a lyophilized form and further including two or more additives selected from the group consisting of surfactants, hygroscopic excipients, and bulking agents; wherein the gelatin comprises about 60% by weight or greater of the total weight of the combination of the gelatin and the polymeric carbohydrate present in the reconstituted composition.

16. The composition according to claim 15, wherein the flowable, injectable reconstituted composition has a flow rate of about 25 μL/s or greater when forced from a syringe through a ⅝ inch long 25 gauge needle under a syringe plunger pressure of 5 N at a temperature of 35° C. to 39° C.

17. The composition according to claim 15, wherein the two or more additives comprise any of: i) a surfactant and a hygroscopic excipient; ii) a surfactant and a bulking agent; iii) a hygroscopic excipient and a bulking agent; iv) a surfactant, a hygroscopic excipient, and a bulking agent; v) a polysorbate and a polyol; vi) a polysorbate and a salt; vii) a polysorbate and a sugar; viii) a polyol and a sugar; ix) a salt and a sugar; x) a polysorbate, a polyol, and a sugar; xi) a polysorbate, a salt, and a sugar; xii) Tween and glycerol; xiii) Tween and NaCl; xiv) Tween and a disaccharide; xv) glycerol and a disaccharide; xvi) NaCl and a disaccharide; xvii) Tween, glycerol, and a disaccharide; and xviii) Tween, NaCl, and a disaccharide.

18. The composition according to claim 15, wherein the two or more additives comprise:
a surfactant and a hygroscopic excipient in a ratio of about 1:20 to about 20:1;
a surfactant and a bulking agent in a ratio of about 1:40 to about 10:1; or
a hygroscopic excipient and a bulking agent in a ratio of about 1:10 to about 10:1.

19. The composition according to claim 15, wherein the flowable, injectable reconstituted composition has a viscosity of about 1.5 Pa-s or less at a temperature of 35° C. to 39° C.

20. The composition according to claim 15, wherein the flowable, injectable reconstituted composition is configured to transition to a solid or semi-solid at temperatures less than 35° C.

21. The composition according to claim 20, wherein the solid or semi-solid reconstituted composition has a residence time in dermal or subdermal tissue of about 3 days or greater.

22. A method of preparing a hydrogel composition, the method comprising:
providing a lyophilized composition comprising gelatin and a polymeric carbohydrate, the gelatin comprising about 60% by weight or greater of the total weight of the combination of the gelatin and the polymeric carbohydrate present in the lyophilized composition;
reconstituting the lyophilized composition with an aqueous reconstitution fluid to form the hydrogel composition such that the reconstituted hydrogel composition includes two or more additives selected from the group consisting of surfactants, hygroscopic excipients, and bulking agents, and such that the total concentration of the gelatin and the polymeric carbohydrate in the reconstituted hydrogel composition is about 50 mg/mL to about 400 mg/mL.

23. The method according to claim 22, wherein one or more of the following conditions is met:
the aqueous reconstitution fluid in contact with the lyophilized composition comprises about 0.01% to about 4% by weight of the surfactant;
the aqueous reconstitution fluid in contact with the lyophilized composition comprises about 0.01% to about 4% by weight of the hygroscopic excipient; and
the aqueous reconstitution fluid in contact with the lyophilized composition comprises about 0.01% to about 4% by weight of the bulking agent.

24. The method according to claim 22, wherein the aqueous reconstitution fluid in contact with the lyophilized composition comprises about 0.05% to about 6% by weight of the additives.

25. The method according to claim 22, wherein the two or more additives comprise any of: i) a surfactant and a hygroscopic excipient; ii) a surfactant and a bulking agent; iii) a hygroscopic excipient and a bulking agent; iv) a surfactant, a hygroscopic excipient, and a bulking agent; v) a polysorbate and a polyol; vi) a polysorbate and a salt; vii) a polysorbate and a sugar; viii) a polyol and a sugar; ix) a salt and a sugar; x) a polysorbate, a polyol, and a sugar; xi) a polysorbate, a salt, and a sugar; xii) Tween and glycerol; xiii) Tween and NaCl; xiv) Tween and a disaccharide; xv) glycerol and a disaccharide; xvi) NaCl and a disaccharide; xvii) Tween, glycerol, and a disaccharide; and xviii) Tween, NaCl, and a disaccharide.

26. The method according to claim 22, wherein the two or more additives comprise:
a surfactant and a hygroscopic excipient in a ratio of about 1:20 to about 20:1;
a surfactant and a bulking agent in a ratio of about 1:40 to about 10:1; or
a hygroscopic excipient and a bulking agent in a ratio of about 1:10 to about 10:1.

27. A kit comprising:
a first container housing a lyophilized composition comprising gelatin and a polymeric carbohydrate, the gelatin comprising about 60% by weight or greater of the total weight of the combination of the gelatin and the polymeric carbohydrate present in the lyophilized composition; and
a second container housing a reconstitution material; and
instructions for combining the contents of the first container and the second container to form a reconstituted hydrogel composition useful for treatment of an acute dermal wound;
wherein the combined contents of the first container and the second container include two or more additives selected from the group consisting of surfactants, hygroscopic excipients, and bulking agents.

28. The kit according to claim 27, wherein the first container includes at least one of the additives.

29. The kit according to claim 27, wherein the second container includes the two or more additives.

30. The kit according to claim 27, wherein the second container includes an aqueous reconstitution fluid.

31. The kit according to claim 27, wherein the aqueous reconstitution fluid includes the two or more additives.

32. The kit according to claim 31, wherein one or more of the following conditions is met:
the aqueous reconstitution fluid comprises about 0.01% to about 4% by weight of the surfactant;
the aqueous reconstitution fluid comprises about 0.01% to about 4% by weight of the hygroscopic excipient; and
the aqueous reconstitution fluid comprises about 0.01% to about 4% by weight of the bulking agent.

33. The kit according to claim 31, wherein the aqueous reconstitution fluid comprises about 0.05% to about 6% by weight of the additives.

34. The kit according to claim 27, wherein the two or more additives comprise any of:
i) a surfactant and a hygroscopic excipient; ii) a surfactant and a bulking agent; iii) a hygroscopic excipient and a bulking agent; iv) a surfactant, a hygroscopic excipient, and a bulking agent; v) a polysorbate and a polyol; vi) a polysorbate and a salt; vii) a polysorbate and a sugar; viii) a polyol and a sugar; ix) a salt and a sugar; x) a polysorbate, a polyol, and a sugar; xi) a polysorbate, a salt, and a sugar; xii) Tween and glycerol; xiii) Tween and NaCl; xiv) Tween and a disaccharide; xv) glycerol and a disaccharide; xvi) NaCl and a disaccharide; xvii) Tween, glycerol, and a disaccharide; and xviii) Tween, NaCl, and a disaccharide.

35. The kit according to claim 27, wherein the two or more additives comprise;
a surfactant and a hygroscopic excipient in a ratio of about 1:20 to about 20:1;
a surfactant and a bulking agent in a ratio of about 1:40 to about 10:1; or a hygroscopic excipient and a bulking agent in a ratio of about 1:10 to about 10:1.

36. The kit according to claim 27, further comprising a connector adapted for one or both of sterile transfer of the contents of one of the first container into the second container and sterile transfer of the contents of the second container into the first container.

37. The kit according to claim 27, wherein at least one of the first container and the second container is a syringe, and wherein the kit optionally comprise a 23 gauge through 27 gauge needle adapted for attachment to the syringe.
